(19) United States
(12) Patent Application Publication
(10) Pub. No.: US 2026/0263491 A1
(43) Pub. Date: Sep. 10, 2026

(54) METHODS AND COMPOSITIONS FOR TREATING LUPUS

(71) Applicant: Ohio State Innovation Foundation, Columbus, OH (US)

(72) Inventor: Wael JARJOUR, Columbus, OH (US)

(21) Appl. No.: 18/871,648

(22) PCT Filed: Jun. 7, 2023
(86) PCT No.: PCT/US2023/024671

§ 371 Date: Dec. 4, 2024

Related U.S. Application Data
(60) Provisional application No. 63/350,182, filed on Jun. 8, 2022.

(51) Int. Cl. A61K 31/69; A61K 31/573; A61P 29/00; A61P 37/02

(52) U.S. Cl. CPC A61K 31/69; A61K 31/573; A61P 29/00; A61P 37/02

(57) ABSTRACT

Disclosed are methods for treating lupus (e.g., systemic lupus erythematosus (SLE)) in a subject using carboranes and carborane analogs. In some embodiments, the carborane or carborane analog comprises a selective ERβ agonist (e.g., a carborane or carborane analog having an ERβ-to-ERα agonist ratio of 8 or more, such as an ERβ-to-ERα agonist ratio of 400 or more). In certain embodiments, the carborane or carborane analog can comprise WT-IV-012. Also provided am pharmaceutical compositions comprising a therapeutically effective amount of a carborane or carborane analog to treat lupus.

### CROSS-REFERENCE TO RELATED APPLICATIONS

This application claims the benefit of priority to U.S. Provisional Application No. 63/350,182 filed Jun. 8, 2022, which is hereby incorporated herein by reference in its entirety.

### BACKGROUND

Lupus is a group of conditions with similar underlying mechanisms involving autoimmunity. In these conditions antibodies created by the body to attack antigens (e.g., viruses, bacteria) became unable to differentiate between antigens and healthy tissue. Thus, the antibodies that should protect the body begin to attack the body's own healthy tissues. Triggers for lupus include viruses, bacteria, hormones (e.g., estrogens), environmental stimulants (e.g., ultraviolet light, sunlight, stress), and certain medications.

Lupus is generally a chronic disease in which the signs and symptoms tend to fluctuate in severity. Common signs or symptoms of lupus include, for example, joint pain and stiffness; muscle aches, pains, or weakness, fever with no other known cause; feeling very tired; butterfly-shaped rash across the nose and cheeks; other skin rashes; sores in mouth or nose, unusual weight loss; anemia; trouble thinking, memory problems, and confusion; kidney problems with no known cause: chest pain when taking a deep breath; sun or light sensitivity; hair loss; and purple or pale fingers or toes from cold or stress. Less common symptoms include blood clots, seizures, strokes, severe headache, dizzy spells, “seeing things” or not being able to judge reality, feeling sad, and dry or irritated eyes. Lupus also increases the risk of developing various other diseases and/or causes other diseases to occur earlier in life. Such diseases include heart disease, osteoporosis, and kidney disease.

Types of lupus include, for example, systemic lupus erythematosus (SLE) including incomplete lupus (subjects who have limited manifestation of the disease) and/or preclinical lupus (subjects with genetic and/or other susceptibility marker but no overt clinical manifestations), cutaneous lupus erythematosus (CLE) (CLE includes, e.g., acute cutaneous lupus erythematosus (ACLE), subacute cutaneous lupus erythematosus (SCLE), intermittent cutaneous lupus erythematosus, and chronic cutaneous lupus), drug-induced lupus, and neonatal lupus. About 70% of all cases of lupus are SLE. CLE can have symptoms that are limited to the skin or can be seen in those with SLE.

Lupus affects predominately young women. More than 90% of people with lupus are women between the ages of 15 and 45. African-American, Latina, Asian, and Native American women are at greater risk of developing lupus than are white women. Men are at higher risk for developing lupus before puberty and after age 50. For African-American women between the ages of 15 and 64, the prevalence is one per 245 women. This prevalence rate for African-American women makes lupus one of the most common chronic diseases of this population.

Accordingly, new methods and compositions for the treatment of lupus are needed.

### SUMMARY

Disclosed herein are methods and compositions for the treatment of lupus (e.g., systemic lupus erythematosus).

Methods for the treatment of lupus can comprise administering a therapeutically effective amount of a carborane or carborane analog to the subject. In some embodiments, these methods can comprise administering a therapeutically effective amount of a carborane. In some embodiments, the carborane can be defined by Formula II, or a pharmaceutically acceptable salts thereof:

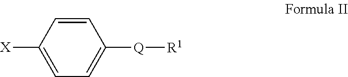

wherein
Q is a substituted or unsubstituted dicarba-closo-dodecaborane cluster, and

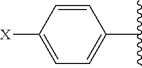

and R¹ are attached to Q in a para configuration;
X is OH, NHR², SH, or S(O)(O)NHR²;
R¹ is substituted or unsubstituted C₄-C₂₀ alkyl, substituted or unsubstituted C₂-C₂₀ alkenyl, substituted or unsubstituted C₂-C₂₀ alkynyl, substituted or unsubstituted C₃-C₂₀ alkylaryl, substituted or unsubstituted C₃-C₂₀ alkylheteroaryl, substituted or unsubstituted C₄-C₂₀ alkylcycloalkyl, substituted or unsubstituted C₄-C₂₀ alkylheterocycloalkyl, substituted or unsubstituted C₁-C₂₀ acyl, or NR³R⁴;
R² is H, OH, halogen, or substituted or unsubstituted C₁-C₄ alkyl;
R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, or substituted or unsubstituted C₁-C₂₀ acyl;
with the proviso that when X is OH, R¹ is not (CH₂)₅CH(CH₃)₂ or NH₂.

In some examples of Formula II, Q can be:

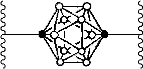

wherein
● is a carbon atom or a boron atom; and
◯ is C—H, C-halogen, C-alkyl, C—OH, C—NH₂, B—H, B-halogen, B-alkyl, B—OH, or B—NH₂.

In some examples of Formula II, X is OH.

In some examples of Formula II, R¹ is a substituted or unsubstituted C₆-C₁₀ alkyl. In some examples of Formula II, R¹ is a C₆-C₁₀ hydroxyalkyl. In some examples of Formula II, R¹ is a C₃-C₁₆ hydroxyalkylaryl. In some examples of Formula II, R¹ is a substituted or unsubstituted branched C₄-C₁₀ alkyl. In some examples of Formula II, R¹ is a branched C₄-C₁₀ hydroxyalkyl.

In some examples, the carborane can defined by Formula III, or a pharmaceutically acceptable salt thereof:

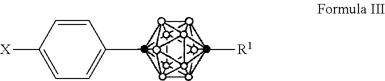

wherein
● is a carbon atom;
◯ is B—H, B-halogen, B-alkyl, B—OH, or B—NH₂;
X is OH, NHR², SH, or S(O)(O)NHR²;
R¹ is substituted or unsubstituted C₄-C₂₀ alkyl, substituted or unsubstituted C₂-C₂₀ alkenyl, substituted or unsubstituted C₂-C₂₀ alkynyl, substituted or unsubstituted C₃-C₂₀ alkylaryl, substituted or unsubstituted C₃-C₂₀ alkylheteroaryl, substituted or unsubstituted C₄-C₂₀ alkylcycloalkyl, substituted or unsubstituted C₄-C₂₀ alkylheterocycloalkyl, substituted or unsubstituted C₁-C₂₀ acyl, or NR³R⁴;
R² is H, OH, halogen, or substituted or unsubstituted C₁-C₄ alkyl; and
R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, or substituted or unsubstituted C₁-C₂₀ acyl;
with the proviso that when X is OH, R¹ is not (CH₂)₅CH(CH₃)₂ or NH₂.

In some examples of Formula III, X is OH.

In some examples, the carborane can be defined by Formula IV, or a pharmaceutically acceptable salt thereof:

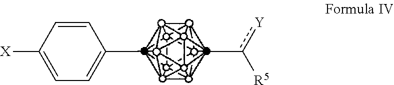

wherein
● is a carbon atom;
◯ is B—H, B-halogen, B-alkyl, B—OH, or B—NH₂;
the dotted line to Y indicates that the bond can be a single bond or a double bond, as valence permits;
X is OH, NHR², SH, or S(O)(O)NHR²;
Y is O, OR², NHR², SH, or S(OXO)NHR²;
R⁵ is substituted or unsubstituted C₂-C₁₉ alkyl, substituted or unsubstituted C₂-C₁₉ alkenyl, substituted or unsubstituted C₂-C₁₉ alkynyl, substituted or unsubstituted C₂-C₁₉ alkylaryl, substituted or unsubstituted C₂-C₁₉ alkylheteroaryl, substituted or unsubstituted C₃-C₁₉ alkylcycloalkyl, substituted or unsubstituted C₃-C₁₉ alkylheterocycloalkyl, or NR³R⁴;
R² is H, OH, halogen, or substituted or unsubstituted C₁-C₄ alkyl;
R^{2′} is H or substituted or unsubstituted C₁-C₄ alkyl; and
R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, or substituted or unsubstituted C₁-C₂₀ acyl.

In some examples of Formula IV, the carborane cluster can include a heteroatom. In some examples of Formula IV, the carborane cluster can include an isotopically labeled atom (i.e., a radiolabeled atom). In some examples of Formula IV, the carborane cluster can include an isotopically labeled Boron atom (e.g., ¹⁰B).

In some examples of Formula IV, X is OH.

In some examples of Formula IV, Y is OH. In some examples of Formula IV, Y is O.

In some examples of Formula IV, R⁵ is a substituted or unsubstituted C₃-C₉ alkyl. In some examples of Formula IV, R⁵ is a substituted or unsubstituted C₆-C₉ alkyl. In some examples of Formula IV, R⁵ is a substituted or unsubstituted C₂-C₁₅ alkylaryl. In some examples of Formula IV, R⁵ is a substituted or unsubstituted branched C₂-C₉ alkyl.

In some embodiments, the carborane or carborane analog comprises an ERs agonist. For example, in some cases, the carborane or carborane analog has an EC₅₀ of 800 nM or less, such as an EC₅₀ of 6 nM or less, against estrogen receptor beta (ERβ).

In certain embodiments, the carborane or carborane analog comprises a selective ERβ agonist. For example, in some cases, the carborane or carborane analog can have an ERβ-to-ERα agonist ratio of 8 or more, such as an ERβ-to-ERα agonist ratio of 400 or more.

In certain embodiments, the carborane or carborane analog can comprise WT-IV-012, the structure of which is shown below.

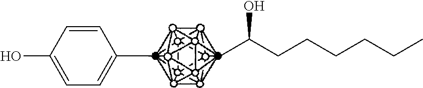

The lupus can comprise, for example, systemic lupus erythematosus (SLE), cutaneous lupus erythematosus (CLE), drug-induced lupus, or neonatal lupus. In certain embodiments, the lupus comprises systemic lupus erythematosus (SLE).

In some cases, the methods can further comprise administration of at least one additional therapeutic as part of the treatment regimen, such as a corticosteroid, an NSAID, an antimalarial, an immunomodulating agent, an immunosuppressive agent, an anticoagulant, or a combination thereof. In certain embodiments, the methods can further comprise administration of a corticosteroid, such as prednisone, prednisolone, methylprednisolone, betamethasone, dexamethasone, triamcinolone, hydrocortisone, or any combination thereof.

Also provided are pharmaceutical compositions comprising a therapeutically effective amount of a carborane or carborane analog described herein (e.g., WT-IV-012) for the treatment of lupus. Optionally, these compositions can further include at least one additional therapeutic as part of the treatment regimen, such as a corticosteroid (e.g., prednisone, prednisolone, methylprednisolone, betamethasone, dexamethasone, triamcinolone, hydrocortisone, or any combination thereof), an NSAID, an antimalarial, an immunomodulating agent, an immunosuppressive agent, an anticoagulant, or a combination thereof.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A-1D illustrate that oral treatment with an example carborane and selective ERβ agonist (WT-IV-012 inhibits kidney nuclear infiltration in SLE Humanized Mice. FIGS. 1A, 1B, and 1C show kidney tissue taken from untreated SLE Humanized Mice (FIG. 1A), SLE Humanized Mice treated with WT-IV-012 (FIG. 1B), and SLE Humanized Mice treated with prednisone (FIG. 1C). FIG. 1D is a plot showing the nuclear pixel count per total area for untreated SLE Humanized Mice, SLE Humanized Mice treated with WT-IV-012, and SLE Humanized Mice treated with prednisone.

FIGS. 2A-2C illustrate that oral treatment with an example carborane and selective ERβ agonist (WT-IV-012 inhibits heart inflammation in SLE Humanized Mice. FIGS. 2A, 2B, and 2C show heart tissue taken from untreated SLE Humanized Mice (FIG. 2A), SLE Humanized Mice treated with WT-IV-012 (FIG. 2B), and SLE Humanized Mice treated with prednisone (FIG. 2C).

FIGS. 3A-3J show the results an MSD cytokine assay comparing the impact of oral treatment with an example carborane and selective ERβ agonist (WT-IV-012, -Erb) or prednisone (-pred) on the circulating level of ten cytokines that are important in inflammatory responses and immune system regulation. The following cytokines were measured: IL-1 (FIG. 3A), IL-2 (FIG. 3B), IL-4 (FIG. 3C), IL-6 (FIG. 3D), IL-8 (FIG. 3E), IL-10 (FIG. 3F), IL-12p70 (FIG. 3G), IL-13 (FIG. 3H), IFNγ (FIG. 3I), and TNFα (FIG. 3J). The cytokine levels were assessed at week 0, week 3, and week 5 following administration.

FIG. 4A is a schematic illustration of a study performed to evaluate the effect of an example carborane and selective ERβ agonist (WT-IV-012) on NFKB activity in NF-kB-LUC reporter mice. FIG. 4B is a photograph showing the result of in vivo imaging system (IVIS) measurements of NF-kB activity in NF-kB-LUC reporter mice treated with WT-IV-012 (left) and vehicle alone (tight). FIG. 4C is a plot comparing NF-kB activity in NF-kB-LUC reporter mice treated with WT-IV-012 and vehicle alone.

### DETAILED DESCRIPTION

The compounds, compositions, and methods described herein may be understood more readily by reference to the following detailed description of specific aspects of the disclosed subject matter and the Examples included therein.

Before the present compounds, compositions, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings.

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the component” includes mixtures of two or more such components, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician.

The term “inhibit” refers to a decrease in an activity, response, condition, disease, or other biological parameter. This can include but is not limited to the complete ablation of the activity, response, condition, or disease. This can also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. By way of example, in the context of lupus, “treating,” “treat,” and “treatment” as used herein, refers to partially or completely inhibiting or reducing lupus from which the subject is suffering. In one embodiment, this term refers to an action that occurs while a patient is suffering from, or is diagnosed with, lupus, which reduces the severity of the condition, or retards or slows the progression of the condition. Treatment need not result in a complete cure of the condition; partial inhibition or reduction of lupus is encompassed by this term.

“Therapeutically effective amount,” as used herein, refers to a minimal amount or concentration of an ERβ agonist that, when administered alone or in combination, is sufficient to provide a therapeutic benefit in the treatment of the condition, or to delay or minimize one or more symptoms associated with the condition. The term “therapeutically effective amount” can encompass an amount that improves overall therapy, reduces or avoids symptoms or causes of the condition, or enhances the therapeutic efficacy of another therapeutic agent. The therapeutic amount need not result in a complete cure of the condition; partial inhibition or reduction of lupus (or a symptom thereof) is encompassed by this term.

As used herein, unless otherwise specified, the terms “prevent,” “preventing” and “prevention” refers to an action that occurs before the subject begins to suffer from the condition, or relapse of such condition. The prevention need not result in a complete prevention of the condition; partial prevention or reduction of lupus (or a symptom thereof) in a subject is encompassed by this term.

As used herein, unless otherwise specified, a “prophylactically effective amount” of an ERβ that, when administered alone or in combination, prevent the condition, or one or more symptoms associated with the condition, or prevent its recurrence. The term “prophylactically effective amount” can encompass an amount that improves overall prophylaxis or enhances the prophylactic efficacy of another prophylactic agent. The prophylactic amount need not result in a complete prevention of the condition; partial prevention or reduction of lupus (or a symptom thereof) in a subject is encompassed by this term. In other embodiments, a “prophylactically effective amount” can refer to an amount that prevents a flare of lupus symptoms in a subject.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

### Chemical Definitions

Terms used herein will have their customary meaning in the art unless specified otherwise. The organic moieties mentioned when defining variable positions within the general formulae described herein (e.g., the term “halogen”) are collective terms for the individual substituents encompassed by the organic moiety. The prefix Cn-Cm preceding a group or moiety indicates, in each case, the possible number of carbon atoms in the group or moiety that follows.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, heteroatoms present in a compound or moiety, such as nitrogen, can have hydrogen substituents and/or any permissible substituents of organic compounds described herein which satisfy the valency of the heteroatom. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound (e.g., a compound that does not spontaneously undergo transformation such as by rearrangement, cyclization, elimination, etc.

“Z¹,” “Z²,” “Z³,” and “Z⁴” are used herein as generic symbols to represent various specific substituents. These symbols can be any substituent, not limited to those disclosed herein, and when they are defined to be certain substituents in one instance, they can, in another instance, be defined as some other substituents.

As used herein, the term “alkyl” refers to saturated, straight-chained or branched saturated hydrocarbon moieties. Unless otherwise specified, C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, or C₁-C₄) alkyl groups are intended. Examples of alkyl groups include methyl, ethyl, propyl, 1-methyl-ethyl, butyl, 1-methyl-propyl, 2-methyl-propyl, 1,1-dimethyl-ethyl, pentyl, 1-methyl-butyl, 2-methyl-butyl, 3-methyl-butyl, 2,2-dimethyl-propyl, 1-ethyl-propyl, hexyl, 1,1-dimethyl-propyl, 1,2-dimethyl-propyl, 1-methyl-pentyl, 2-methyl-pentyl, 3-methyl-pentyl, 4-methyl-pentyl, 1,1-dimethyl-butyl, 1,2-dimethyl-butyl, 1,3-dimethyl-butyl, 2,2-dimethyl-butyl, 2,3-dimethyl-butyl, 3,3-dimethyl-butyl, 1-ethyl-butyl, 2-ethyl-butyl, 1,1,2-trimethyl-propyl, 1,2,2-trimethyl-propyl, 1-ethyl-1-methyl-propyl, and 1-ethyl-2-methyl-propyl. Alkyl substituents may be unsubstituted or substituted with one or more chemical moieties. The alkyl group can be substituted with one or more groups including, but not limited to, hydroxy, halogen, acyl, alkyl, alkoxy, alkenyl, alkynyl, aryl, heteroaryl, acyl, aldehyde, amino, carboxylic acid, ester, ether, ketone, nitro, silyl, sulfo-oxo, sulfonyl, sulfone, sulfoxide, or thiol, as described below, provided that the substituents are sterically compatible and the rules of chemical bonding and strain energy are satisfied. The alkyl group can also include one or more heteroatoms (e.g., from one to three heteroatoms) incorporated within the hydrocarbon moiety. Examples of heteroatoms include, but are not limited to, nitrogen, oxygen, sulfur, and phosphorus.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halides (halogens; e.g., fluorine, chlorine, bromine, or iodine). The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

As used herein, the term “alkenyl” refers to unsaturated, straight-chained, or branched hydrocarbon moieties containing a double bond. Unless otherwise specified, C₂-C₂₄ (e.g., C₂-C₂₂, C₂-C₂₀, C₂-C₁₈, C₂-C₁₆, C₂-C₁₄, C₂-C₁₂, C₂-C₁₀, C₂-C₈, C₂-C₆, C₂-C₄) alkenyl groups are intended. Alkenyl groups may contain more than one unsaturated bond. Examples include ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, and 1-ethyl-2-methyl-2-propenyl. The term “vinyl” refers to a group having the structure —CH═CH₂; 1-propenyl refers to a group with the structure —CH═CH—CH₃; and 2-propenyl refers to a group with the structure —CH₂—CH═CH₂. Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C. Alkenyl substituents may be unsubstituted or substituted with one or more chemical moieties. Examples of suitable substituents include, for example, alkyl, halogenated alkyl, alkoxy, alkenyl, alkynyl, aryl, heteroaryl, acyl, aldehyde, amino, carboxylic acid, ester, ether, halide, hydroxy, ketone, nitro, silyl, sulfo-oxo, sulfonyl, sulfone, sulfoxide, or thiol, as described below, provided that the substituents are sterically compatible and the rules of chemical bonding and strain energy are satisfied.

As used herein, the term “alkynyl” represents straight-chained or branched hydrocarbon moieties containing a triple bond. Unless otherwise specified, C₂-C₂₄ (e.g., C₂-C₂₂, C₂-C₂₀, C₂-C₁₈, C₂-C₁₆, C₂-C₁₄, C₂-C₁₂, C₂-C₁₀, C₂-C₈, C₂-C₆, C₂-C₄) alkynyl groups are intended. Alkynyl groups may contain more than one unsaturated bond. Examples include C₂-C₆-alkynyl, such as ethynyl, 1-propynyl, 2-propynyl (or propargyl), 1-butynyl, 2-butynyl, 3-butynyl, 1-methyl-2-propynyl, 1-pentynyl, 2-pentynyl, 3-pentynyl, 4-pentynyl, 3-methyl-1-butynyl, 1-methyl-2-butynyl, 1-methyl-3-butynyl, 2-methyl-3-butynyl, 1,1-dimethyl-2-propynyl, 1-ethyl-2-propynyl, 1-hexynyl, 2-hexynyl, 3-hexynyl, 4-hexynyl, 5-hexynyl, 3-methyl-1-pentynyl, 4-methyl-1-pentynyl, 1-methyl-2-pentynyl, 4-methyl-2-pentynyl, 1-methyl-3-pentynyl, 2-methyl-3-pentynyl, 1-methyl-4-pentynyl, 2-methyl-4-pentynyl, 3-methyl-4-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, I-ethyl-2-butynyl, 1-ethyl-3-butynyl, 2-ethyl-3-butynyl, and 1-ethyl-1-methyl-2-propynyl. Alkynyl substituents may be unsubstituted or substituted with one or more chemical moieties. Examples of suitable substituents include, for example, alkyl, halogenated alkyl, alkoxy, alkenyl, alkynyl, aryl, heteroaryl, acyl, aldehyde, amino, carboxylic acid, ester, ether, halide, hydroxy, ketone, nitro, silyl, sulfo-oxo, sulfonyl, sulfone, sulfoxide, or thiol, as described below.

As used herein, the term “aryl,” as well as derivative terms such as aryloxy, refers to groups that include a monovalent aromatic carbocyclic group of from 3 to 20 carbon atoms. Aryl groups can include a single ring or multiple condensed rings. In some embodiments, aryl groups include C₆-C₁₀ aryl groups. Examples of aryl groups include, but are not limited to, phenyl, biphenyl, naphthyl, tetrahydronaphthyl, phenylcyclopropyl, and indanyl. In some embodiments, the aryl group can be a phenyl, indanyl or naphthyl group. The term “heteroaryl” is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is included in the term “aryl,” defines a group that contains an aromatic group that does not contain a heteroatom. The aryl or heteroaryl substituents may be unsubstituted or substituted with one or more chemical moieties. Examples of suitable substituents include, for example, alkyl, halogenated alkyl, alkoxy, alkenyl, alkynyl, aryl, heteroaryl, acyl, aldehyde, amino, carboxylic acid, cycloalkyl, ester, ether, halide, hydroxy, ketone, nitro, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, acyl, aldehyde, amino, carboxylic acid, ester, ether, halide, hydroxy, ketone, nitro, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, acyl, aldehyde, amino, carboxylic acid, ester, ether, halide, hydroxy, ketone, nitro, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

As used herein, “heteroaryl” refers to a monocyclic or polycyclic aromatic heterocycle having at least one heteroatom ring member selected from sulfur, oxygen, and nitrogen. In some embodiments, the heteroaryl ring has 1, 2, 3, or 4 heteroatom ring members independently selected from nitrogen, sulfur and oxygen. In some embodiments, any ring-forming N in a heteroaryl moiety can be an N-oxide. In some embodiments, the heteroaryl has 5-10 ring atoms and 1, 2, 3 or 4 heteroatom ring members independently selected from nitrogen, sulfur and oxygen. In some embodiments, the heteroaryl has 5-6 ring atoms and 1 or 2 heteroatom ring members independently selected from nitrogen, sulfur and oxygen. In some embodiments, the heteroaryl is a five-membered or six-membered heteroaryl ring. A five-membered heteroaryl ring is a heteroaryl with a ring having five ring atoms wherein one or more (e.g., 1, 2, or 3) ring atoms are independently selected from N, O, and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered heteroaryl ring is a heteroaryl with a ring having six ring atoms wherein one or more (e.g., 1, 2, or 3) ring atoms are independently selected from N, O, and S. Exemplary six-membered ring heteroaryls are pyridyl, pyrazinyl, pyrimidinyl, triazinyl and pyridazinyl.

As used herein, “heterocycloalkyl” refers to non-aromatic monocyclic or polycyclic heterocycles having one or more ring-forming heteroatoms selected from O, N, or S. Included in heterocycloalkyl are monocyclic 4-, 5-, 6-, and 7-membered heterocycloalkyl groups. Heterocycloalkyl groups can also include spirocycles. Example heterocycloalkyl groups include pyrrolidin-2-one, 1,3-isoxazolidin-2-one, pyranyl, tetrahydropuran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, and the like. Ring-forming carbon atoms and heteroatoms of a heterocycloalkyl group can be optionally substituted by oxo or sulfido (e.g., C(O), S(O), C(S), or S(O)₂, etc.). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the cycloalkyl ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl has 4-10, 4-7 or 4-6 ring atoms with 1 or 2 heteroatoms independently selected from nitrogen, oxygen, or sulfur and having one or more oxidized ring members.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

The term “acyl” as used herein is represented by the formula —C(O)Z¹ where Z¹ can be a hydrogen, hydroxyl, alkoxy, alkyl, halogenated alkyl, alkenyl, alkynyl, aryl, heteroaryl, cycloalkyl, cycloalkenyl, heterocycloalkyl, or heterocycloalkenyl group described above. As used herein, the term “acyl” can be used interchangeably with “carbonyl.” Throughout this specification “C(O)” or “CO” is a short hand notation for C═O.

As used herein, the term “alkoxy” refers to a group of the formula Z¹—O—, where Z¹ is unsubstituted or substituted alkyl as defined above. Unless otherwise specified, alkoxy groups wherein Z¹ is a C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, C₁-C₄) alkyl group are intended. Examples include methoxy, ethoxy, propoxy, 1-methyl-ethoxy, butoxy, 1-methyl-propoxy, 2-methyl-propoxy, 1,1-dimethyl-ethoxy, pentoxy, 1-methyl-butyloxy, 2-methyl-butoxy, 3-methyl-butoxy, 2,2-di-methyl-propoxy, 1-ethyl-propoxy, hexoxy, 1,1-dimethyl-propoxy, 1,2-dimethyl-propoxy, 1-methyl-pentoxy, 2-methyl-pentoxy, 3-methyl-pentoxy, 4-methyl-penoxy, 1,1-dimethyl-butoxy, 1,2-dimethyl-butoxy, 1,3-dimethyl-butoxy, 2,2-dimethyl-butoxy, 2,3-dimethyl-butoxy, 3,3-dimethyl-butoxy, 1-ethyl-butoxy, 2-ethylbutoxy, 1,1,2-trimethyl-propoxy, 1,2,2-trimethyl-propoxy, 1-ethyl-1-methyl-propoxy, and 1-ethyl-2-methyl-propoxy.

The term “aldehyde” as used herein is represented by the formula —C(O)H.

The terms “amine” or “amino” as used herein are represented by the formula —NZ¹Z², where Z¹ and Z² can each be substitution group as described herein, such as hydrogen, an alkyl, halogenated alkyl, alkenyl, alkynyl, aryl, heteroaryl, cycloalkyl, cycloalkenyl, heterocycloalkyl, or heterocycloalkenyl group described above. “Amido” is —C(O)NZ¹Z².

The term “carboxylic acid” as used herein is represented by the formula —C(O)OH. A “carboxylate” or “carboxyl” group as used herein is represented by the formula —C(O)O⁻.

The term “ester” as used herein is represented by the formula —OC(O)Z¹ or —C(O)OZ¹, where Z¹ can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula Z¹OZ², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula Z¹C(O)Z², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “halide” or “halogen” or “halo” as used herein refers to fluorine, chlorine, bromine, and iodine.

The term “hydroxyl” as used herein is represented by the formula —OH.

The term “nitro” as used herein is represented by the formula —NO₂.

The term “silyl” as used herein is represented by the formula —SiZ¹Z²Z³, where Z¹, Z², and Z³ can be, independently, hydrogen, alkyl, halogenated alkyl, alkoxy, alkenyl, alkynyl, aryl, heteroaryl, cycloalkyl, cycloalkenyl, heterocycloalkyl, or heterocycloalkenyl group described above.

The term “sulfonyl” is used herein to refer to the sulfo-oxo group represented by the formula —S(O)₂Z¹, where Z¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “sulfonylamino” or “sulfonamide” as used herein is represented by the formula —S(O)₂NH—.

The term “thiol” as used herein is represented by the formula —SH.

The term “thio” as used herein is represented by the formula —S—.

As used herein, Me refers to a methyl group; OMe refers to a methoxy group; and i-Pr refers to an isopropyl group.

“R¹,” “R²,” “R³,” “Rⁿ,” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an amine group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

Unless stated to the contrary, a formula with chemical bonds shown only as solid lines and not as wedges or dashed lines contemplates each possible stereoisomer or mixture of stereoisomer (e.g., each enantiomer, each diastereomer, each meso compound, a racemic mixture, or scalemic mixture).

Reference will now be made in detail to specific aspects of the disclosed materials, compounds, compositions, articles, and methods, examples of which are illustrated in the accompanying Examples and Figures.

### Carboranes and Carborane Analogs

Dicarba-closo-dodecaborane (also referred to herein as “carborane”) is an icosahedral cluster containing two carbon atoms and ten boron atoms in which both atoms are hexacoordinated. In carboranes, depending on the position of the carbon atoms in the cluster, 3 kinds of isomers exist, i.e., 1,2-dicarba-closo-dodecaborane (ortho-carborane), 1,7-dicarba-closo-dodecaborane (meta-carborane), and 1,12-dicarba-closo-dodecaborane (para-carborane). These structures are unique among boron compounds, as they can have high thermal stabilities and hydrophobicities, for example, comparable to hydrocarbons.

Carboranes can be used, for example, in ¹⁰Boron-Neutron Capture Therapy (BNCT). BNCT has been developed as a therapy for glioma and melanoma. When ¹⁰B is irradiated with thermal neutron (slow neutron), and a ray with 2.4 MeV energy is emitted and the atom decomposed to ⁷Li and ⁴He. The range of a ray is about 10 μm, which corresponds to the diameter of cells Therefore, effects are expected that only cells in which ¹⁰B atoms are uptaken are destroyed and other cells are not damaged. For the development of BNCT, it is important to have cancer cells selectively uptake ¹⁰B atoms in a concentration capable of destroying cells with neutron radiation. For that purpose, other-carborane skeleton has been utilized which has been utilized which has low toxicity and a high ¹⁰B content, and is easy to be synthesized. Moreover, nucleic acid precursors, amino acids, and porphyrins which contain ortho-carboranes have been synthesized and subjected to evaluation.

Carborane-based ERβ agonists and carborane analogs are described, for example, in U.S. Pat. No. 6,838,574 to Endo, U.S. Patent Application Publication No. 2018/0264017 to Tjarks et al., and PCT/US2019/064228 to Coss et al., and PCT/US2021/021909 to Bansal et al., each of which is hereby incorporated by reference in its entirety.

In some embodiments, the carborane can be defined by Formula I below:

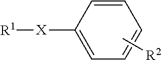

wherein
R¹ represents a dicarba-closo-dodecaboran-yl group which may have one or more substituents selected from the group consisting of an alkyl group, an alkenyl group, a carboxyl group, an alkoxycarbonyl group, an amino group, a hydroxyl group, a hydroxyalkyl group, a mono or di-alkylcarbamoyl-substituted alkyl group, an alkanoyl group, an aryl group, and an aralkyl group, each of which may be substituted or unsubstituted;
R² represents a carboxyl group, an alkoxycarbonyl group, or a hydroxyl group;
X represents a single bond, or a linking group selected from the group consisting of groups represented by the following formulas:

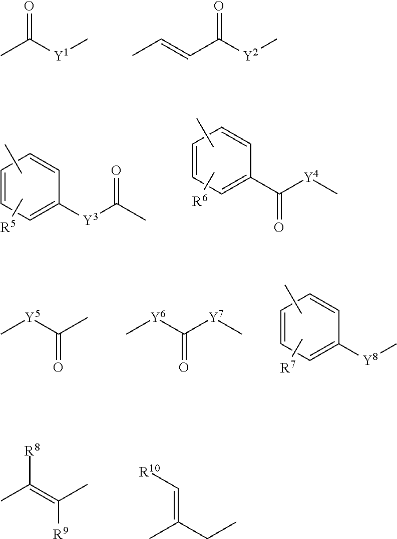

wherein Y¹, Y², Y³, Y⁴, Y⁵, Y⁶, and Y⁷ independently represent an oxygen atom or —N(R³)— wherein R³ represents hydrogen atom or an alkyl group; Y⁸ represents an oxygen atom, —N(R⁴)— wherein R⁴ represents hydrogen atom or an alkyl group, —CO—, —CH₂—, or —C(═CH²)—; R⁵, R⁶, and R⁷ independently represent hydrogen or one or more substituents on the phenyl group; R⁸ represents an alkyl group or an aryl group which may be substituted; R⁹ represents an alkyl group; and R¹⁰ represents a substituted or unsubstituted aryl group.

In some embodiments, the carborane can be defined by Formula II, or a pharmaceutically acceptable salts thereof:

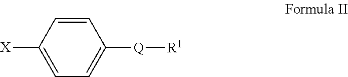

wherein
Q is a substituted or unsubstituted dicarba-closo-dodecaborane cluster, and

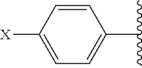

and R¹ are attached to Q in a para configuration;
X is OH, NHR², SH, or S(O)(O)NHR²;
R¹ is substituted or unsubstituted C₄-C₂₀ alkyl, substituted or unsubstituted C₂-C₂₀ alkenyl, substituted or unsubstituted C₂-C₂₀ alkynyl, substituted or unsubstituted C₃-C₂₀ alkylaryl, substituted or unsubstituted C₃-C₂₀ alkylheteroaryl, substituted or unsubstituted C₄-C₂₀ alkylcycloalkyl, substituted or unsubstituted C₄-C₂₀ alkylheterocycloalkyl, substituted or unsubstituted C₁-C₂₀ acyl, or NR³R⁴;
R² is H, OH, halogen, or substituted or unsubstituted C₁-C₄ alkyl;
R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, or substituted or unsubstituted C₁-C₂₀ acyl;
with the proviso that when X is OH, R¹ is not (CH₂)₅CH(CH₃)₂ or NH₂.

In some examples of Formula II, the carborane cluster can include a heteroatom. In some examples of Formula II, the carborane cluster can include an isotopically labeled atom (i.e., a radiolabeled atom). In some examples of Formula II, the carborane cluster can include an isotopically labeled Boron atom (e.g., ¹⁰B).

In some examples of Formula II, Q can be:

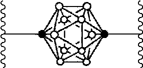

wherein
● is a carbon atom or a boron atom; and
◯ is C—H, C-halogen, C-alkyl, C—OH, C—NH₂, B—H, B-halogen, B-alkyl, B—OH, or B—NH₂.

In some examples of Formula II, X is OH.

In some examples of Formula II, R¹ is a substituted or unsubstituted C₆-C₁₀ alkyl. In some examples of Formula II, R¹ is a C₆-C₁₀ hydroxyalkyl. In some examples of Formula II, R¹ is a substituted or unsubstituted C₃-C₁₆ alkylaryl. In some examples of Formula II, R¹ is a C₃-C₁₆ hydroxyalkylaryl. In some examples of Formula II, R¹ is a substituted or unsubstituted C₅-C₁₀ acyl. In some examples of Formula II, R¹ is a substituted or unsubstituted branched C₄-C₁₀ alkyl.

In some examples of Formula II, R¹ is a branched C₄-C₁₀ hydroxyalkyl.

In some examples of Formula II, the compounds can be of Formula III, or a pharmaceutically acceptable salt thereof:

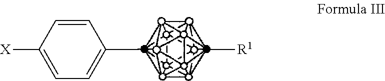

wherein
● is a carbon atom;
◯ is B—H, B-halogen, B-alkyl, B—OH, or B—NH₂;
X is OH, NHR², SH, or S(O)(O)NHR²;
R¹ is substituted or unsubstituted C₄-C₂₀ alkyl, substituted or unsubstituted C₂-C₂₀ alkenyl, substituted or unsubstituted C₂-C₂₀ alkynyl, substituted or unsubstituted C₃-C₂₀ alkylaryl, substituted or unsubstituted C₃-C₂₀ alkylheteroaryl, substituted or unsubstituted C₄-C₂₀ alkylcycloalkyl, substituted or unsubstituted C₄-C₂₀ alkylheterocycloalkyl, substituted or unsubstituted C₁-C₂₀ acyl, or NR³R⁴;
R² is H, OH, halogen, or substituted or unsubstituted C₁-C₄ alkyl; and
R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, or substituted or unsubstituted C₁-C₂₀ acyl;
with the proviso that when X is OH, R¹ is not (CH₂)₅CH(CH₃)₂ or NH₂.

In some examples of Formula III, the carborane cluster can include a heteroatom.

In some examples of Formula III, the carborane cluster can include an isotopically labeled atom (i.e., a radiolabeled atom). In some examples of Formula III, the carborane cluster can include an isotopically labeled Boron atom (e.g., ¹⁰B).

In some examples of Formula III, X is OH.

In some examples of Formula III, R¹ is a substituted or unsubstituted C₆-C₁₀ alkyl. In some examples of Formula III, R¹ is a C₆-C₁₀ hydroxyalkyl. In some examples of Formula III, R¹ is a substituted or unsubstituted C₃-C₁₆ alkylaryl. In some examples of Formula III, R¹ is a C₃-C₁₆ hydroxyalkylaryl. In some examples of Formula III, R¹ is a substituted or unsubstituted C₅-C₁₀ acyl. In some examples of Formula III, R¹ is a substituted or unsubstituted branched C₄-C₁₀ alkyl. In some examples of Formula III, R¹ is a branched C₄-C₁₀ hydroxyalkyl.

In some examples of Formula III, the compounds can be of Formula IV, or a pharmaceutically acceptable salt thereof.

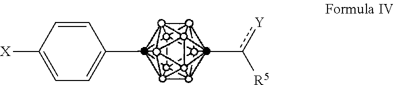

wherein
● is a carbon atom;
◯ is B—H, B-halogen, B-alkyl, B—OH, or B—NH₂;
the dotted line to Y indicates that the bond can be a single bond or a double bond, as valence permits;
X is OH, NHR², SH, or S(O)(O)NHR²;
Y is O, OR², NHR², SH, or S(OXO)NHR²;
R⁵ is substituted or unsubstituted C₂-C₁₉ alkyl, substituted or unsubstituted C₂-C₁₉ alkenyl, substituted or unsubstituted C₂-C₁₉ alkynyl, substituted or unsubstituted C₂-C₁₉ alkylaryl, substituted or unsubstituted C₂-C₁₉ alkylheteroaryl, substituted or unsubstituted C₃-C₁₉ alkylcycloalkyl, substituted or unsubstituted C₃-C₁₉ alkylheterocycloalkyl, or NR³R⁴;
R² is H, OH, halogen, or substituted or unsubstituted C₁-C₄ alkyl;
R^{2′} is H or substituted or unsubstituted C₁-C₄ alkyl; and
R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, or substituted or unsubstituted C₁-C₂₀ acyl.

In some examples of Formula IV, the carborane cluster can include a heteroatom. In some examples of Formula IV, the carborane cluster can include an isotopically labeled atom (i.e., a radiolabeled atom). In some examples of Formula IV, the carborane cluster can include an isotopically labeled Boron atom (e.g., ¹⁰B).

In some examples of Formula IV, X is OH.

In some examples of Formula IV, Y is OH. In some examples of Formula IV, Y is O.

In some examples of Formula IV, R⁵ is a substituted or unsubstituted C₃-C₉ alkyl. In some examples of Formula IV, R⁵ is a substituted or unsubstituted C₆-C₉ alkyl. In some examples of Formula IV, R⁵ is a substituted or unsubstituted C₂-C₁₈ alkylaryl. In some examples of Formula IV, R⁵ is a substituted or unsubstituted branched C₂-C₉ alkyl.

Also disclosed herein are compounds of Formula V, and pharmaceutically acceptable salts thereof.

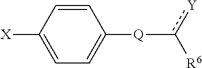

wherein
Q is a substituted or unsubstituted dicarba-closo-dodecaborane cluster, and

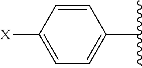

and

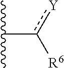

and are attached to Q in a para configuration;
the dotted line to Y indicates that the bond can be a single bond or a double bond, as valence permits;
X is OH, NHR², SH, or S(O)(O)NHR²;
Y is O, OR^{2′}, NHR², SH, or S(OXO)NHR²;
R⁶ is substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₂-C₂₀ alkylheteroaryl, substituted or unsubstituted C₄-C₂₀ alkylcycloalkyl, substituted or unsubstituted C₄-C₂₀ alkylheterocycloalkyl, or NR³R⁴;
R² is H, OH, halogen, or substituted or unsubstituted C₁-C₄ alkyl;
R^{2′} is H or substituted or unsubstituted C₁-C₄ alkyl; and
R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, or substituted or unsubstituted C₁-C₂₀ acyl;
with the proviso that when X is OH, R⁶ is not CH₂OH, CH(CH₃)OH, CH₂CH₂OH, CH₂CH₂CH₂OH, (CH₂)₅CH(CH₃)₂, or NH₂.

In some examples of Formula V, the carborane cluster can include a heteroatom. In some examples of Formula V, the carborane cluster can include an isotopically labeled atom (i.e., a radiolabeled atom). In some examples of Formula V, the carborane cluster can include an isotopically labeled Boron atom (e.g., ¹⁰B).

In some examples of Formula V, Q can be:

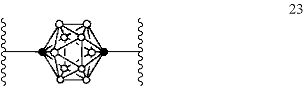

wherein
● is a carbon atom or a boron atom; and
◯ is C—H, C-halogen, C-alkyl, C—OH, C—NH₂, B—H, B-halogen, B-alkyl, B—OH, or B—NH₂.

In some examples of Formula V, X is OH.

In some examples of Formula V, Y is OH. In some examples of Formula V, Y is O.

In some examples of Formula V, R⁶ is a substituted or unsubstituted C₆-C₁₀ alkyl. In some examples of Formula V, R⁶ is a substituted or unsubstituted C₂-C₁₅ alkylaryl. In some examples of Formula V, R⁶ is a substituted or unsubstituted branched C₃-C₁₀ alkyl.

In some examples of Formula V, the compounds can be of Formula VI, or a pharmaceutically acceptable salt thereof:

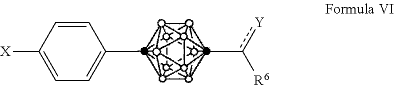

wherein
● is a carbon atom;
◯ is B—H, B-halogen, B-alkyl, B—OH, or B—NH₂;
the dotted line to Y indicates that the bond can be a single bond or a double bond, as valence permits;
X is OH, NHR², SH, or S(O)(O)NHR²;
Y is O, OR², NHR², SH, or S(OXO)NHR²;
R⁶ is substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₂-C₂₀ alkylheteroaryl, substituted or unsubstituted C₄-C₂₀ alkylcycloalkyl, substituted or unsubstituted C₄-C₂₀ alkylheterocycloalkyl, or NR³R⁴;
R² is H, OH, halogen, or substituted or unsubstituted C₁-C₄ alkyl;
R^{2′} is H or substituted or unsubstituted C₁-C₄ alkyl; and
R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, or substituted or unsubstituted C₁-C₂₀ acyl;
with the proviso that when X is OH, R⁶ is not CH₂OH, CH(CH₃)OH, CH₂CH₂OH, CH₂CH₂CH₂OH, (CH₂)₅CH(CH₃)₂, or NH₂.

In some examples of Formula VI, the carborane cluster can include a heteroatom. In some examples of Formula VI, the carborane cluster can include an isotopically labeled atom (i.e., a radiolabeled atom). In some examples of Formula VI, the carborane cluster can include an isotopically labeled Boron atom (e.g., ¹⁰B).

In some examples of Formula VI, X is OH.

In some examples of Formula VI, Y is OH. In some examples of Formula VI, Y is O.

In some examples of Formula VI, R⁶ is a substituted or unsubstituted C₆-C₁₀ alkyl. In some examples of Formula VI, R⁶ is a substituted or unsubstituted C₂-C₁₈ alkylaryl. In some examples of Formula VI, R⁶ is a substituted or unsubstituted branched C₃-C₁₀ alkyl.

Also disclosed herein are compounds of Formula VII, and pharmaceutically acceptable salts thereof.

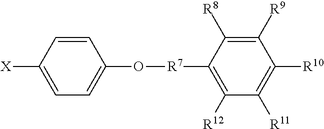

wherein
Q is a substituted or unsubstituted dicarba-closo-dodecaborane cluster, and

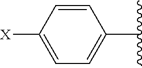

and R⁷ are attached to Q in a para configuration;
X is OH, NHR², SH, or S(O)(O)NHR²;
R⁷ is substituted or unsubstituted C₁-C₁₄ alkyl, substituted or unsubstituted C₂-C₁₄ alkenyl, substituted or unsubstituted C₂-C₁₄ alkynyl, substituted or unsubstituted C₁-C₁₄ acyl, or NR³R⁴;
R⁸, R⁹, R¹⁰, R¹¹, and R¹² are independently H, OH, halogen, substituted or unsubstituted C₁-C₂₀ alkyl, sub substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, substituted or unsubstituted C₁-C₂₀ acyl, or NR³R⁴, or wherein, as valence permits, R⁸ and R⁹, R⁹ and R¹⁰, R¹⁰ and R¹¹, or R¹¹ and R¹², together with the atoms to which they are attached, form a 3-10 membered substituted or unsubstituted cyclic moiety optionally including from 1 to 3 heteroatoms;
R² is H, OH, halogen, or substituted or unsubstituted C₁-C₄ alkyl; and
R¹ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, or substituted or unsubstituted C₁-C₂₀ acyl.

In some examples of Formula VII, the carborane cluster can include a heteroatom. In some examples of Formula VII, the carborane cluster can include an isotopically labeled atom (i.e., a radio labeled atom). In some examples of Formula VII, the carborane cluster can include an isotopically labeled Boron atom (e.g., ¹⁰B).

In some examples of Formula VII, Q can be:

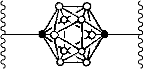

wherein
● is a carbon atom or a boron atom; and
◯ is C—H, C-halogen, C-alkyl, C—OH, C—NH₂, B—H, B-halogen, B-alkyl, B—OH, or B—NH₂.

In some examples of Formula VII, X is OH.

In some examples of Formula VII, R⁷ is a substituted or unsubstituted C₁-C₇ alkyl. In some examples of Formula VII, R⁷ is a C₁-C₇ hydroxyalkyl.

In some examples of Formula VII, R⁸-R¹² are independently H, OH, halogen, or substituted or unsubstituted C₁-C₄ alkyl, or wherein, as valence permits, R⁸ and R⁹, R⁹ and R¹⁰, R¹⁰ and R¹¹, or R¹¹ and R¹², together with the atoms to which they are attached, form a 3-10 membered substituted or unsubstituted cyclic moiety optionally including from 1 to 3 heteroatoms. In some examples of Formula VII, R⁸-R¹² are each H. In some examples of Formula VII, R⁸, R¹⁰, and R¹² are each H, and R⁹ and R¹⁰, together with the atoms to which they are attached, form a substituted or unsubstituted 5-7 membered cyclic moiety.

In some examples of Formula VII, the compounds can be of Formula VIII, or a pharmaceutically acceptable salt thereof:

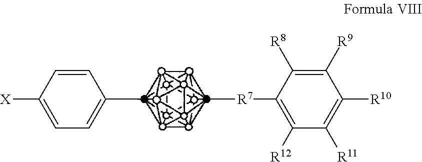

wherein
● is a carbon atom;
◯ is B—H, B-halogen, B-alkyl, B—OH, or B—NH₂;
X is OH, NHR², SH, or S(O)(O)NHR²;
R⁷ is substituted or unsubstituted C₁-C₁₄ alkyl, substituted or unsubstituted C₂-C₁₄ alkenyl, substituted or unsubstituted C₂-C₁₄ alkynyl, substituted or unsubstituted C₁-C₁₄ acyl, or NR³R⁴;
R⁸, R⁹, R¹⁰, R¹¹, and R¹² are independently H, OH, halogen, substituted or unsubstituted C₁-C₂₀ alkyl, sub substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, substituted or unsubstituted C₁-C₂₀ acyl, or NR³R⁴, or wherein, as valence permits, R⁸ and R⁹, R⁹ and R¹⁰, R¹⁰ and R¹¹, or R¹¹ and R¹², together with the atoms to which they are attached, form a 3-10 membered substituted or unsubstituted cyclic moiety optionally including from 1 to 3 heteroatoms;
R² is H, OH, halogen, or substituted or unsubstituted C₁-C₄ alkyl; and
R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, or substituted or unsubstituted C₁-C₂₀ acyl.

In some examples of Formula VIII, the carborane cluster can include a heteroatom. In some examples of Formula VIII, the carborane cluster can include an isotopically labeled atom (i.e., a radiolabeled atom). In some examples of Formula VII, the carborane cluster can include an isotopically labeled Boron atom (e.g., ¹⁰B).

In some examples of Formula VIII, X is OH.

In some examples of Formula VIII, R⁷ is a substituted or unsubstituted C₁-C₇ alkyl. In some examples of Formula VIII, R⁷ is a C₁-C₇ hydroxyalkyl.

In some examples of Formula VIII, R⁸-R¹² are independently H, OH, halogen, or substituted or unsubstituted C₁-C₄ alkyl, or wherein, as valence permits, R⁸ and R⁹, R⁹ and R¹⁰, R¹⁰ and R¹¹, or R¹¹ and R¹², together with the atoms to which they are attached, form a 3-10 membered substituted or unsubstituted cyclic moiety optionally including from 1 to 3 heteroatoms. In some examples of Formula VIII, R⁸-R¹² are each H. In some examples of Formula VIII, R⁸, R¹⁰, and R¹² are each H, and R⁹ and R¹⁰, together with the atoms to which they are attached, form a substituted or unsubstituted 5-7 membered cyclic moiety.

Also disclosed herein are compounds of Formula IX, and pharmaceutically acceptable salts thereof;

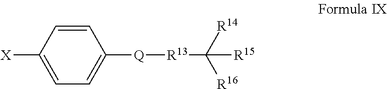

wherein
Q is a substituted or unsubstituted dicarba-closo-dodecaborane cluster, and

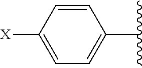

and R¹³ are attached to Q in a para configuration;
X is OH, NHR², SH, or S(O)(O)NR²;
R¹³ is substituted or unsubstituted C₁-C₁₉ alkyl, substituted or unsubstituted C₂-C₁₉ alkenyl, substituted or unsubstituted C₂-C₁₉ alkynyl, or substituted or unsubstituted C₁-C₂₀ acyl; and
R¹⁴, R¹⁵, and R¹⁶ are independently hydrogen, halogen, hydroxyl, substituted or unsubstituted C₁-C₁₈ alkyl, substituted or unsubstituted C₂-C₁₈ alkenyl, substituted or unsubstituted C₁-C₁₈ alkynyl, substituted or unsubstituted C₂-C₁₈ aryl, substituted or unsubstituted C₃-C₁₈ cycloalkyl, substituted or unsubstituted C₁-C₂₀ acyl, or NR³R⁴, or wherein, as valence permits, R¹⁴ and R¹⁵, R¹⁴ and R¹⁶, or R¹⁵ and R¹⁶, together with the atoms to which they are attached, for a 3-10 membered substituted or unsubstituted cyclic moiety optionally including from 1 to 3 heteroatoms,
with the proviso that at least two of R¹⁴, R¹⁵ and R¹⁶ are not hydrogen, halogen, or hydroxyl; and
with the proviso that when X is OH and R³ is a C₅ alkyl, R¹⁴, R¹⁵, and R¹⁶ are not H, methyl, and methyl.

In some examples of Formula IX, the carborane cluster can include a heteroatom. In some examples of Formula IX, the carborane cluster can include an isotopically labeled atom (i.e., a radio labeled atom). In some examples of Formula IX, the carborane cluster can include an isotopically labeled Boron atom (e.g., ¹⁰B). In some examples of Formula IX, Q is:

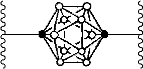

wherein
● is a carbon atom or a boron atom; and
◯ is C—H, C-halogen, C-alkyl, C—OH, C—NH₂, B—H, B-halogen, B-alkyl, B—OH, or B—NH₂.

In some examples of Formula IX, X is OH.

In some examples of Formula IX, R³ is a substituted or unsubstituted C₄-C₈ alkyl. In some examples of Formula IX, R¹³ is a C₄-C₈ hydroxyalkyl.

In some examples of Formula IX, R¹⁴-R¹⁶ are independently hydrogen, halogen, hydroxyl, substituted or unsubstituted C₁-C₄ alkyl, with the proviso that at least two of R¹⁴, R¹⁵ and R¹⁶ are not hydrogen, halogen, or hydroxyl; and with the proviso that when X is OH and R¹³ is a C₅ alkyl, R¹⁴, R¹⁵, and R¹⁶ are not H, methyl, and methyl.

In some examples of Formula IX, the compounds can be of Formula X, or a pharmaceutically acceptable salt thereof:

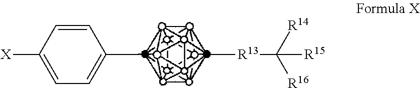

wherein
● is a carbon atom;
◯ is B—H, B-halogen, B-alkyl, B—OH, or B—NH₂;
X is OH, NHR², SH, or S(O)(O)NHR²;
R¹³ is substituted or unsubstituted C₁-C₁₉ alkyl, substituted or unsubstituted C₂-C₁₉ alkenyl, substituted or unsubstituted C₂-C₁₉ alkynyl, or substituted or unsubstituted C₁-C₂₀ acyl; and
R¹⁴, R^{1S}, and R¹⁶ are independently hydrogen, halogen, hydroxyl, substituted or unsubstituted C₁-C₁₈ alkyl, substituted or unsubstituted C₂-C₁₈ alkenyl, substituted or unsubstituted C₁-C₁₈ alkynyl, substituted or unsubstituted C₂-C₁₈ aryl, substituted or unsubstituted C₃-C₁₈ cycloalkyl, substituted or unsubstituted C₁-C₂₀ acyl, or NR³R⁴, or wherein, as valence permits, R¹⁴ and R¹⁵, R¹⁴ and R¹⁶, or R¹⁵ and R¹⁶, together with the atoms to which they are attached, for a 3-10 membered substituted or unsubstituted cyclic moiety optionally including from 1 to 3 heteroatoms,
with the proviso that at least two of R¹⁴, R¹⁵ and R¹⁶ are not hydrogen, halogen, or hydroxyl; and
with the proviso that when X is OH and R¹³ is a C₅ alkyl, R¹⁴, R¹⁵, and R¹⁶ are not H, methyl, and methyl.

In some examples of Formula X, the carborane cluster can include a heteroatom. In some examples of Formula X, the carborane cluster can include an isotopically labeled atom (i.e., a radio labeled atom). In some examples of Formula X, the carborane cluster can include an isotopically labeled Boron atom (e.g., ¹⁰B).

In some examples of Formula X, X is OH.

In some examples of Formula X, R¹³ is a substituted or unsubstituted C₄-C₈ alkyl. In some examples of Formula X, R¹³ is a C₄-C₈ hydroxyalkyl.

In some examples of Formula X, R¹⁴-R¹⁶ are independently hydrogen, halogen, hydroxyl, substituted or unsubstituted C₁-C₄ alkyl, with the proviso that at least two of R¹⁴, R¹⁵ and R¹⁶ are not hydrogen, halogen, or hydroxyl; and with the proviso that when X is OH and R¹³ is a C₅ alkyl, R¹⁴, R¹⁵, and R¹⁶ are not H, methyl, and methyl.

In some examples, the compounds can be selected from the group consisting of:

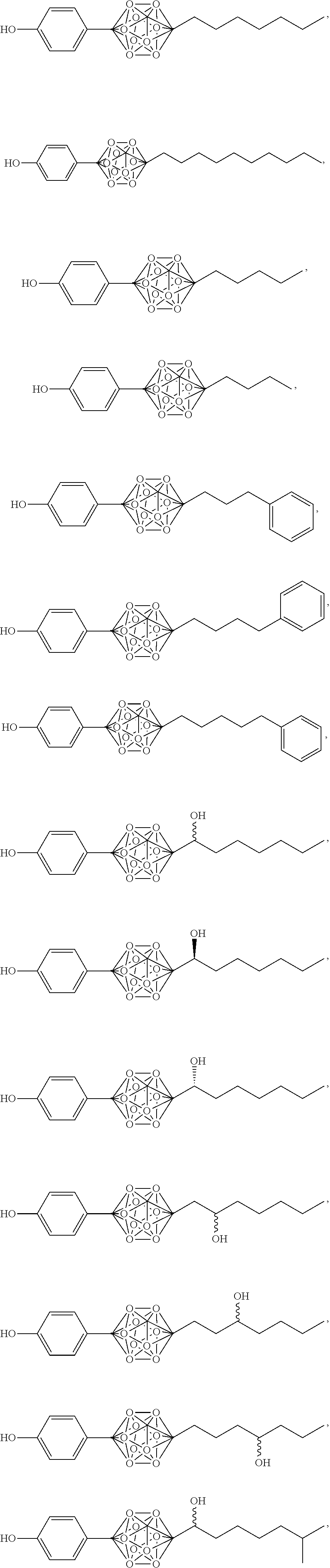

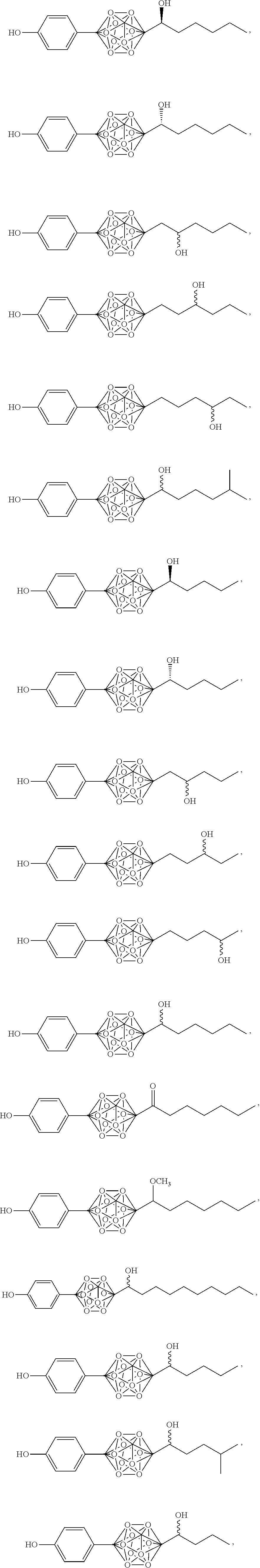

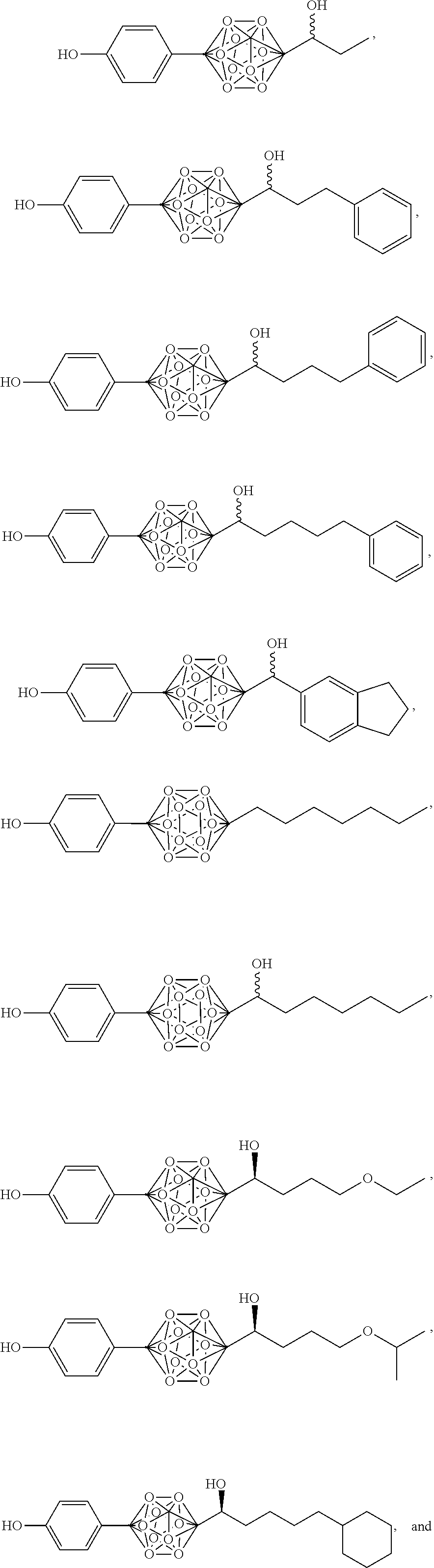

pharmaceutically acceptable salts thereof. In some examples, the carborane cluster can include a heteroatom.

Also disclosed herein are compounds of Formula XI, and pharmaceutically acceptable salts thereof:

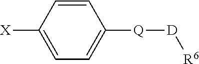

wherein
Q is a substituted or unsubstituted dicarba-closo-dodecaborane cluster;
D is —S—, —S(O)—, —S(O)(O)—, —S(O)(NH)—, —P(O)(OH)O—, —P(O)(OH)NH—, or —O—;
X is OH, NHR², SH, or S(O)(O)NHR²,
R⁶ is substituted or unsubstituted C₁-C₂M alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₂-C₂₀ alkylheteroaryl, substituted or unsubstituted C₄-C₂₀ alkylcycloalkyl, or substituted or unsubstituted C₄-C₂₀ alkylheterocycloalkyl; and
R² is H, OH, halogen, or substituted or unsubstituted C₁-C₄ alkyl.

In some examples of Formula XI,

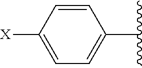

and

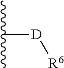

are attached to Q in a para configuration.

In some examples of Formula XI, the carborane cluster can include a heteroatom. In some examples of Formula XI, the carborane cluster can include an isotopically labeled atom (i.e., a radiolabeled atom). In some examples of Formula XI, the carborane cluster can include an isotopically labeled Boron atom (e.g., ¹⁰B).

In some examples of Formula XI, Q can be:

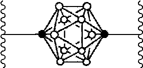

wherein
● is a carbon atom or a boron atom; and
◯ is C—H, C-halogen, C-alkyl, C—OH, C—NH₂, B—H, B-halogen, B-alkyl, B—OH, or B—NH₂.

In some examples of Formula XI, X is OH.

In some examples of Formula XI, R⁶ is a substituted or unsubstituted C₆-C₁₀ alkyl. In some examples of Formula XI, R⁶ is a substituted or unsubstituted C₂-C₁₅ alkylaryl. In some examples of Formula XI, R⁶ is a substituted or unsubstituted branched C₃-C₁₀ alkyl.

In some examples, the compounds can be selected from the group consisting of:

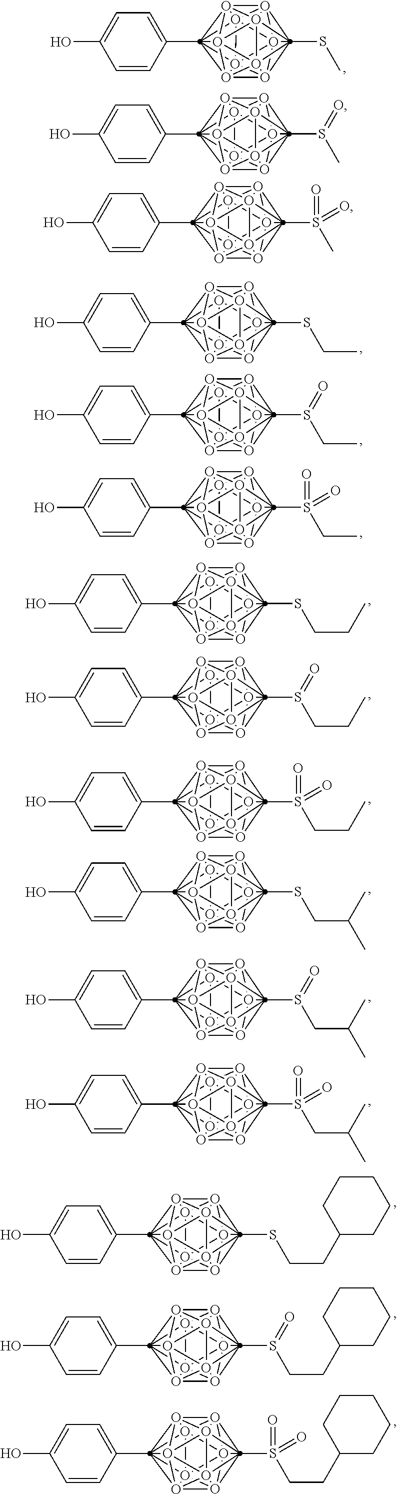

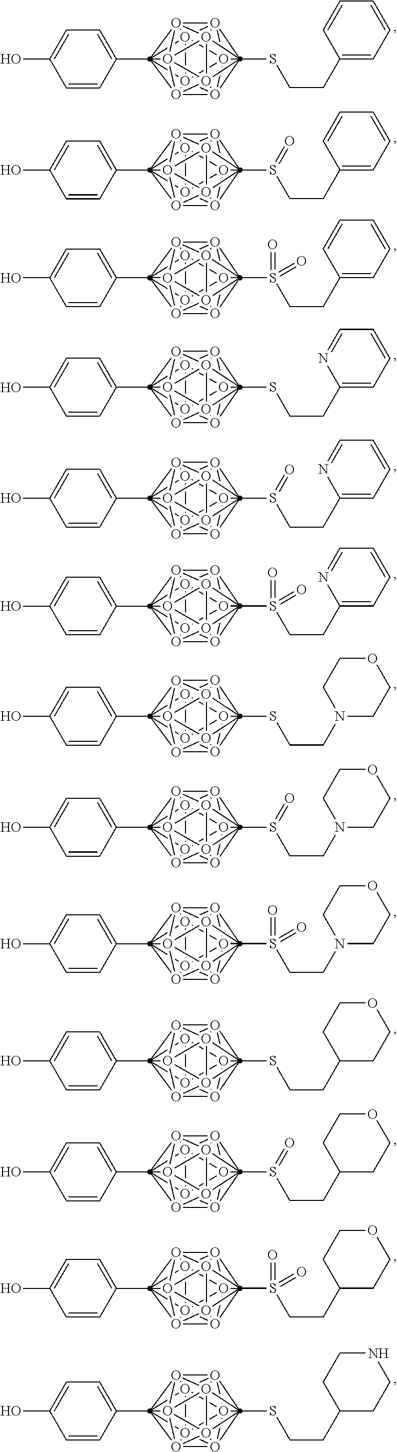

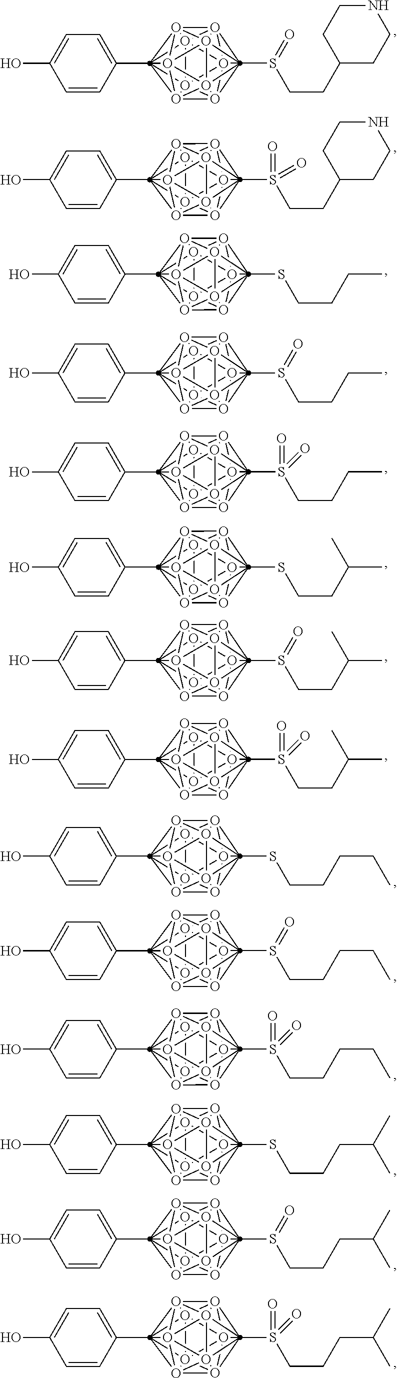

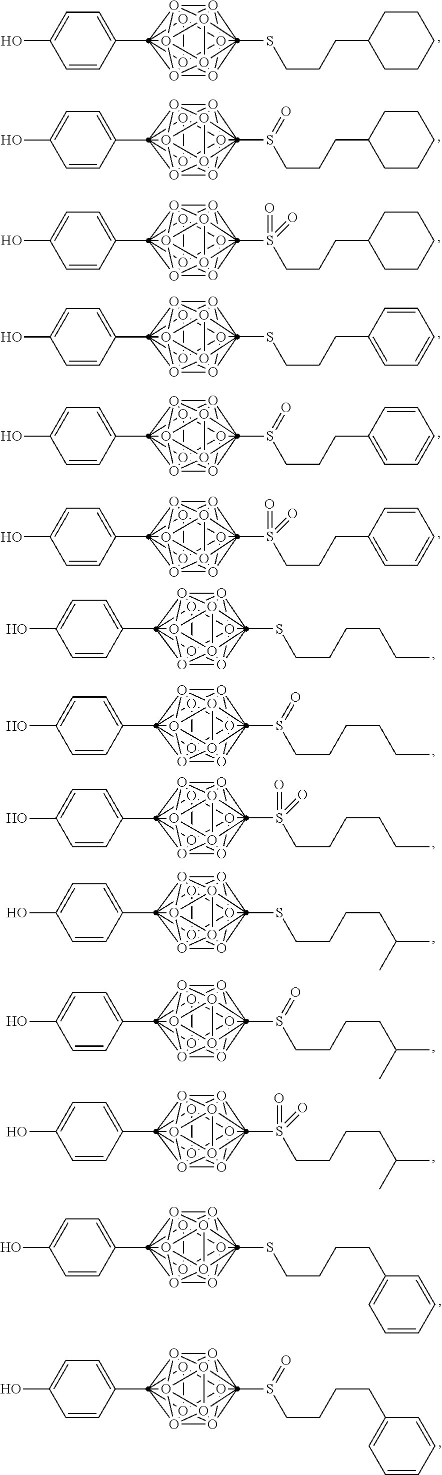

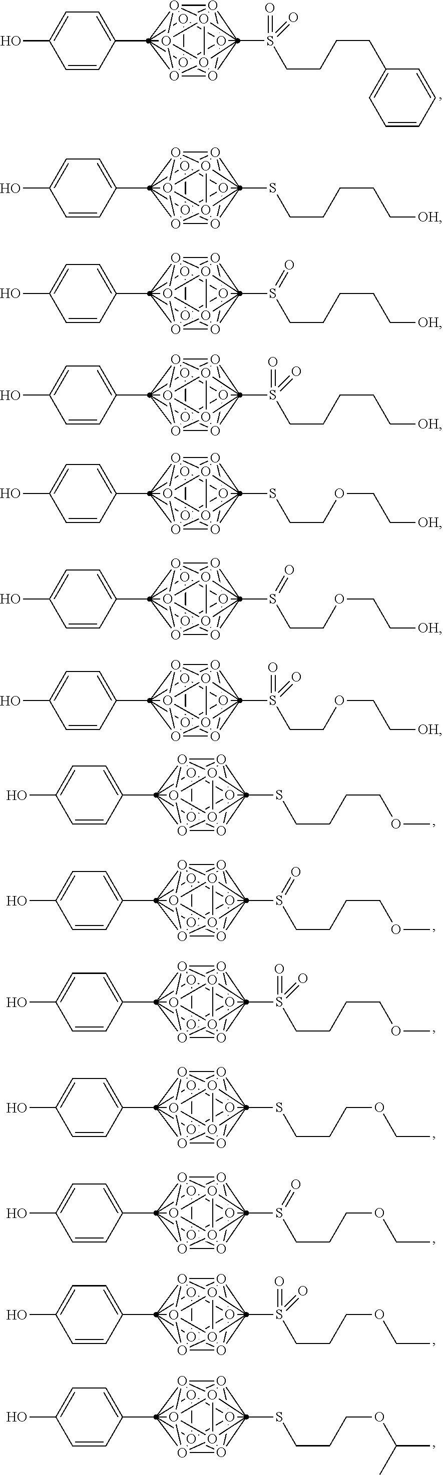

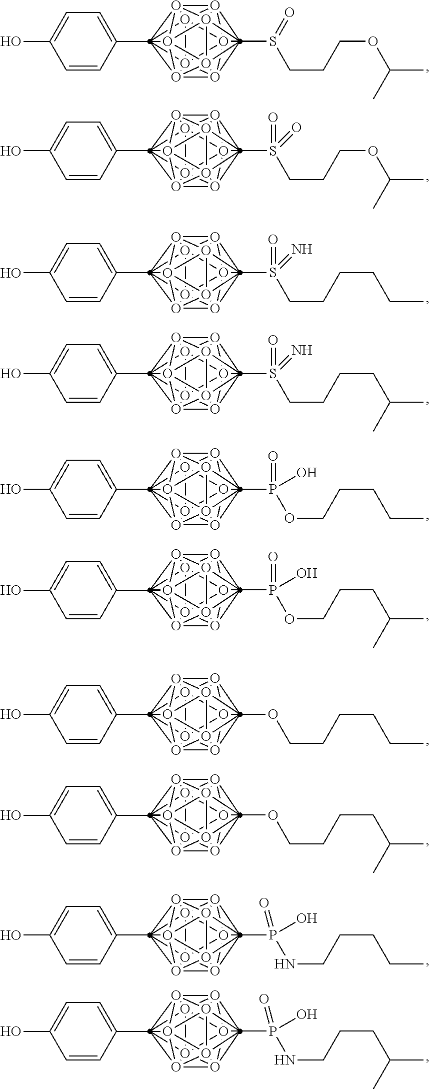

and pharmaceutically acceptable salts thereof. In some examples, the carborane cluster can include a heteroatom.

In some embodiments, the carborane can be defined by Formula XII, or a pharmaceutically acceptable salt thereof:

A-Q-R¹ Formula XII

wherein Q is a substituted or unsubstituted dicarba-closo-dodecaborane cluster, and A and R¹ are attached to Q in a para configuration; A is a substituted or unsubstituted heteroaryl ring; R¹ is substituted or unsubstituted C₂-C₂₀ alkyl, substituted or unsubstituted C₂-C₂₀ alkenyl, substituted or unsubstituted C₂-C₂₀ alkynyl, substituted or unsubstituted C₃-C₂₀ alkylaryl, substituted or unsubstituted C₃-C₂₀ alkylheteroaryl, substituted or unsubstituted C₄-C₂₀ alkylcycloalkyl, substituted or unsubstituted C₄-C₂₀ alkylheterocycloalkyl, substituted or unsubstituted C₁-C₂₀ acyl, C₁-C₂₀ acyl, —C(O)NR³R⁴, —S(O)—R³, —S(O₂)—R³, substituted or unsubstituted C₂-C₂₀ heteroalkyl, or NR³R⁴; and R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, and substituted or unsubstituted C₂-C₂₀ heteroalkyl.

In some embodiments, Q is:

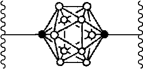

wherein ● is a carbon atom or a boron atom; and ◯ is C—H, C-halogen, C-alkyl, C—OH, C—NH₂, B—H, B-halogen, B-alkyl, B—OH, or B—NH₂.

In some embodiments, A can be a five-membered substituted or unsubstituted heteroaryl ring. For example, A can comprise a thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, or 1,3,4-oxadiazolyl ring. In some embodiments, A can be a six-membered substituted or unsubstituted heteroaryl ring. For example, A can comprise a pyridyl, pyrazinyl, pyrimidinyl, triazinyl, or pyridazinyl ring.

In some cases, the compound can be defined by Formula XIIA, or a pharmaceutically acceptable salt thereof:

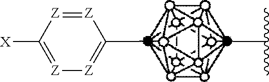

wherein ● is a carbon atom; ◯ is B—H, B-halogen, B-alkyl, B—OH, or B—NH₂; X is OH, NHR², SH, or S(O)(O)NHR²; Z is, individually for each occurrence, N or CH, with the proviso that at least one of Z is N; R¹ is substituted or unsubstituted C₂-C₂₀ alkyl, substituted or unsubstituted C₂-C₂₀ alkenyl, substituted or unsubstituted C₂-C₂₀ alkynyl, substituted or unsubstituted C₃-C₂₀ alkylaryl, substituted or unsubstituted C₃-C₂₀ alkylheteroaryl, substituted or unsubstituted C₄-C₂₀ alkylcycloalkyl, substituted or unsubstituted C₄-C₂₀ alkylheterocycloalkyl, substituted or unsubstituted C₁-C₂₀ acyl, C₁-C₂₀ acyl, —C(O)NR³R⁴, —S(O)—R³, —S(O₂)—R³, substituted or unsubstituted C₂-C₂₀ heteroalkyl, or NR³R⁴; R² is H, OH, halogen, or substituted or unsubstituted C₁-C₄ alkyl; and R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, and substituted or unsubstituted C₂-C₂₀ heteroalkyl.

In some cases, one of Z can be N. In some cases, two or more of Z can be N. In some cases, three of Z can be N.

In some embodiments, the compound can be defined by one of the formulae below, or a pharmaceutically acceptable salt thereof:

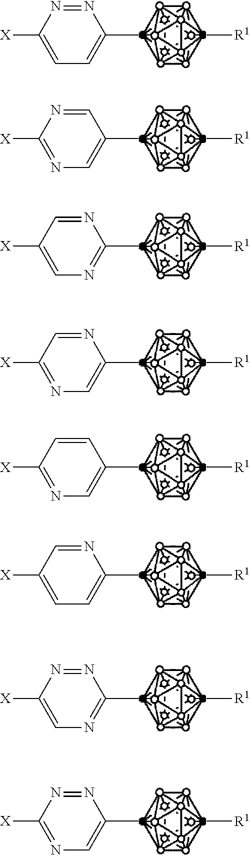

wherein ● is a carbon atom, ◯ is B—H, B-halogen, B-alkyl, B—OH, or B—NH₂; X is OH, NHR², SH or S(O)(O)NHR²; R¹ is substituted or unsubstituted C₂-C₂₀ alkyl, substituted or unsubstituted C₂-C₂₀ alkenyl, substituted or unsubstituted C₂-C₂₀ alkynyl, substituted or unsubstituted C₃-C₂₀ alkylaryl, substituted or unsubstituted C₃-C₂₀ alkylheteroaryl, substituted or unsubstituted C₄-C₂₀ alkylcycloalkyl, substituted or unsubstituted C₄-C₂₀ alkylheterocycloalkyl, substituted or unsubstituted C₁-C₂₀ acyl, C₁-C₂₀ acyl, —C(O)NR³R⁴, —S(O)—R³, —S(O₂)—R³, substituted or unsubstituted C₂-C₂₀ heteroalkyl, or NR³R⁴; R² is H, OH, halogen, or substituted or unsubstituted C₁-C₄ alkyl; and R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, and substituted or unsubstituted C₂-C₂₀ heteroalkyl.

In some embodiments, the compound can be defined by one of Formula XIIB-XIIF, or a pharmaceutically acceptable salt thereof:

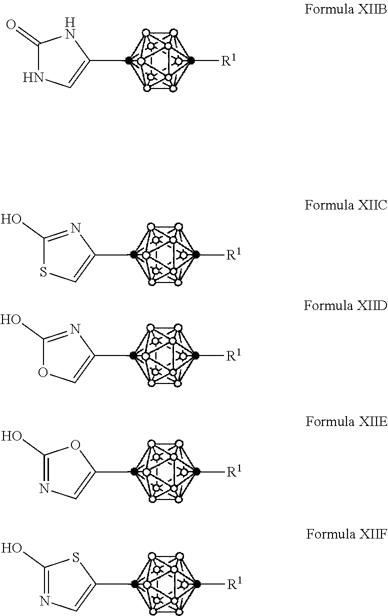

wherein ● is a carbon atom; ◯ is B—H, B-halogen, B-alkyl, B—OH, or B—NH₂; R¹ is substituted or unsubstituted C₂-C₂₀ alkyl, substituted or unsubstituted C₂-C₂₀ alkenyl, substituted or unsubstituted C₂-C₂₀ alkynyl, substituted or unsubstituted C₃-C₂₀ alkylaryl, substituted or unsubstituted C₃-C₂₀ alkylheteroaryl, substituted or unsubstituted C₄-C₂₀ alkylcycloalkyl, substituted or unsubstituted C₄-C₂₀ alkylheterocycloalkyl, substituted or unsubstituted C₁-C₂₀ acyl, C₁-C₂₀ acyl, —C(O)NR³R⁴, —S(O)—R³, —S(O₂)—R³, substituted or unsubstituted C₂-C₂₀ heteroalkyl, or NR³R⁴; R² is H, OH, halogen, or substituted or unsubstituted C₁-C₄ alkyl; and R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, and substituted or unsubstituted C₂-C₂₀ heteroalkyl.

In some of the embodiments above, X can be OH.

In some of the embodiments above, R⁷ can be a substituted or unsubstituted C₆-C₁₀ alkyl (e.g., a C₆-C₁₀ hydroxyalkyl).

In some of the embodiments above, R¹ can be a substituted or unsubstituted C₃-C₁₆ alkylaryl (e.g., a C₃-C₁₆ hydroxyalkylaryl).

In some of the embodiments above, R¹ can be a substituted or unsubstituted C₈-C₂₀ alkylaryl (e.g., a C₅-C₂₀ hydroxyalkylaryl).

In some of the embodiments above, R¹ can be a substituted or unsubstituted C₅-C₁₀ acyl.

In some of the embodiments above, R¹ can be a substituted or unsubstituted branched C₄-C₁₀ alkyl (e.g., a branched C₄-C₁₀ hydroxyalkyl).

In some embodiments, the compound is defined by a formula below, or a pharmaceutically acceptable salt thereof:

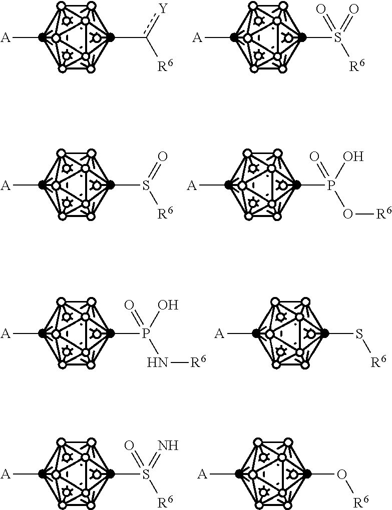

wherein ● is a carbon atom; ◯ is B—H, B-halogen, B-alkyl, B—OH, or B—NH₂; the dotted line to Y indicates that the bond can be a single bond or a double bond, as valence permits; A is a substituted or unsubstituted heteroaryl ring; Y, when present, is O, halogen, OR^{2′}, NHR², SH, or S(O)(O)NHR²; R^{F} is substituted or unsubstituted C₁-C₁₉ alkyl, substituted or unsubstituted C₂-Cig alkenyl, substituted or unsubstituted C₂-C₁₉ alkynyl, substituted or unsubstituted C₂-C₁₉ alkylaryl, substituted or unsubstituted C₂-C₁₉ alkylheteroaryl, substituted or unsubstituted C₄-C₁₉ alkylcycloalkyl, substituted or unsubstituted C₄-C₁₉ alkylheterocycloalkyl, and substituted or unsubstituted C₂-C₂₀ heteroalkyl, or NR³R⁴; R² is H, OH, halogen, or substituted or unsubstituted C₁-C₄ alkyl; R^{2′} is H or substituted or unsubstituted C₁-C₄ alkyl; and R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, and substituted or unsubstituted C₂-C₂₀ heteroalkyl.

In some embodiments, A can be a five-membered substituted or unsubstituted heteroaryl ring. For example, A can comprise a thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, or 1,3,4-oxadiazolyl ring. In some embodiments, A can be a six-membered substituted or unsubstituted heteroaryl ring. For example, A can comprise a pyridyl, pyrazinyl, pyrimidinyl, triazinyl, or pyridazinyl ring.

In some of these embodiments, Y is OH. In some of these embodiments, Y is F. In some of these embodiments, Y is O.

In some examples, R⁷ can be a substituted or unsubstituted C₃-C₁₀ alkyl, such as a substituted or unsubstituted C₆-C₉ alkyl.

In some examples, R⁶ can be a substituted or unsubstituted C₂-C₁₅ alkylaryl.

In some examples, R⁶ can be a substituted or unsubstituted branched C₂-C₉ alkyl.

In some examples, R⁶ can be a substituted or unsubstituted C₃-C₁₀ heteroalkyl, such as a substituted or unsubstituted C₆-C₉ heteroalkyl.

Also provided are compounds defined by Formula XIII, or a pharmaceutically acceptable salt thereof:

A-Q-R¹ Formula XIII

wherein Q is a substituted or unsubstituted dicarba-closo-dodecaborane cluster, and A and R¹ are attached to Q in a para configuration; A is a substituted or unsubstituted aryl ring or a substituted or unsubstituted heteroaryl ring; R¹ is substituted or unsubstituted C₂-C₂₀ heteroalkyl, —C(O)NR³R⁴, —S(O)—R³, —S(O₂)—R³, or NR³R⁴; and R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₂-C₂₀ alkylheteroaryl, substituted or unsubstituted C₄-C₂₀ alkylcycloalkyl, substituted or unsubstituted C₄-C₂₀ alkylheterocycloalkyl, and substituted or unsubstituted C₂-C₂₀ heteroalkyl, with the proviso that when present, at least one of R³ and R⁴ is C₂-C₂₀ heteroalkyl.

In some embodiments, A can comprise a substituted or unsubstituted aryl ring (e.g., a substituted or unsubstituted phenyl ring). In some embodiments, A can be a five-membered substituted or unsubstituted heteroaryl ring. For example, A can comprise a thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, or 1,3,4-oxadiazolyl ring. In some embodiments, A can be a six-membered substituted or unsubstituted heteroaryl ring. For example, A can comprise a pyridyl, pyrazinyl, pyrimidinyl, triazinyl, or pyridazinyl ring.

In some embodiments, Q is:

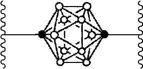

wherein ● is a carbon atom or a boron atom; and ◯ is C—H, C-halogen, C-alkyl, C—OH, C—NH₂, B—H, B-halogen, B-alkyl, B—OH, or B—NH₂.

In some embodiments, the compound can be defined by Formula XIIIA, or a pharmaceutically acceptable salt thereof:

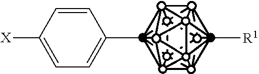

wherein ● is a carbon atom; ◯ is B—H, B-halogen, B-alkyl, B—OH, or B—NH₂; X is OH, NHR², SH, or S(O)(O)NHR²; Z is, individually for each occurrence, N or CH, with the proviso that at least one of Z is N; R¹ is substituted or unsubstituted C₂-C₂₀ heteroalkyl, —C(O)NR³R⁴, —S(O)—R³, —S(O₂)—R³, or NR³R⁴; and R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₂-C₂₀ alkylheteroaryl, substituted or unsubstituted C₄-C₂₀ alkylcycloalkyl, substituted or unsubstituted C₄-C₂₀ alkylheterocycloalkyl, and substituted or unsubstituted C₂-C₂₀ heteroalkyl, with the proviso that when present, at least one of R³ and R⁴ is C₂-C₂₀ heteroalkyl.

In some of these embodiments, X can be OH.

Also provided are compounds defined by any of the formula below, or a pharmaceutically acceptable salt thereof:

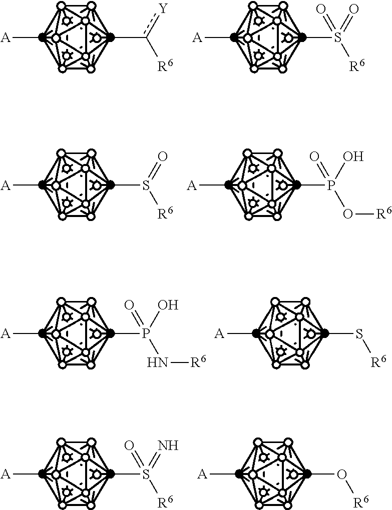

wherein ● is a carbon atom; ◯ is B—H, B-halogen, B-alkyl, B—OH, or B—NH₂; the dotted line to Y indicates that the bond can be a single bond or a double bond, as valence permits; A is a substituted or unsubstituted aryl ring a substituted or unsubstituted heteroaryl ring; Y, when present, is O, halogen, OR², NHR², SH, or S(OXO)NHR²; R⁶ is substituted or unsubstituted C₁-C₁₉ alkyl, substituted or unsubstituted C₂-C₁₉ alkenyl, substituted or unsubstituted C₂-C₁₉ alkynyl, substituted or unsubstituted C₂-C₁₉ alkylaryl, substituted or unsubstituted C₂-C₁₉ alkylheteroaryl, substituted or unsubstituted C₄-C₁₉ alkylcycloalkyl, substituted or unsubstituted C₄-C₁₉ alkylheterocycloalkyl, and substituted or unsubstituted C₂-C₂₀ heteroalkyl, or NR³R⁴; R² is H, OH, halogen, or substituted or unsubstituted C₁-C₄ alkyl; R^{2′} is H or substituted or unsubstituted C₁-C₄ alkyl; and R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, and substituted or unsubstituted C₂-C₂₀ heteroalkyl.

In some embodiments, A can comprise a substituted or unsubstituted aryl ring (e.g., a substituted or unsubstituted phenyl ring). In some embodiments, A can be a five-membered substituted or unsubstituted heteroaryl ring. For example, A can comprise a thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, or 1,3,4-oxadiazolyl ring. In some embodiments, A can be a six-membered substituted or unsubstituted heteroaryl ring. For example, A can comprise a pyridyl, pyrazinyl, pyrimidinyl, triazinyl, or pyridazinyl ring.

In some of these embodiments, Y is OH. In some of these embodiments, Y is F. In some of these embodiments, Y is O.

In some examples, R⁶ can be a substituted or unsubstituted C₃-C₁₀ alkyl, such as a substituted or unsubstituted C₆-C₉ alkyl.

In some examples, R⁶ can be a substituted or unsubstituted C₂-C₁₅ alkylaryl.

In some examples, R⁶ can be a substituted or unsubstituted branched C₂-C₉ alkyl.

In some examples, R⁶ can be a substituted or unsubstituted C₃-C₁₀ heteroalkyl, such as a substituted or unsubstituted C₆-C₉ heteroalkyl.

In some examples, the carborane can be selected from the group consisting of:

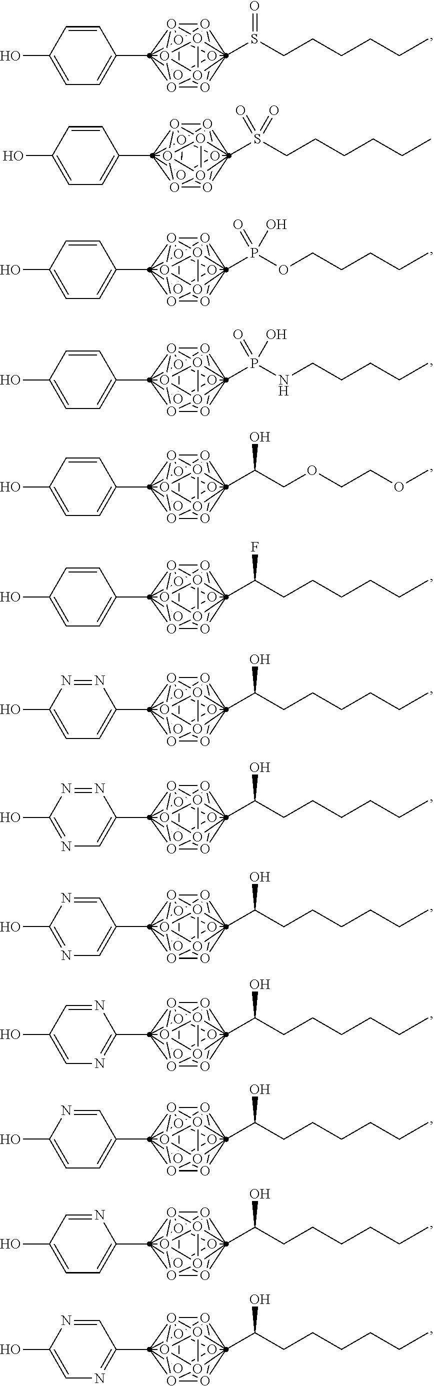

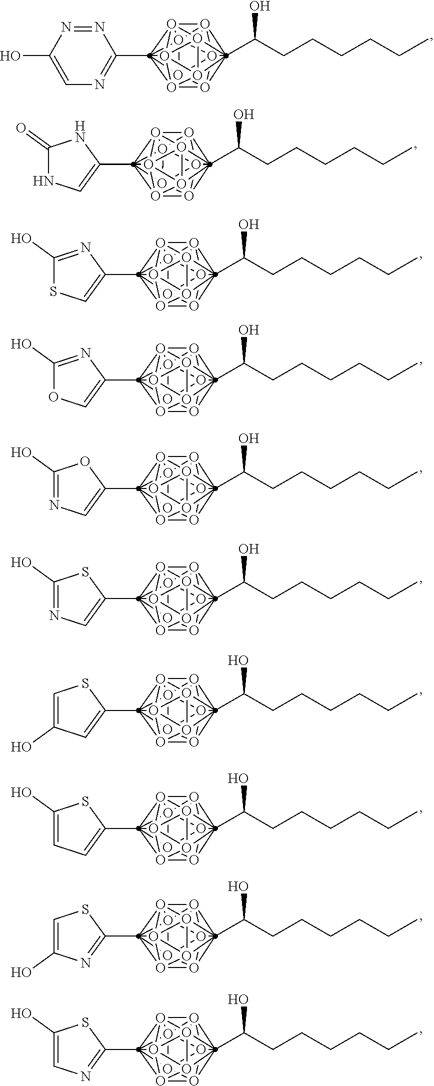

and pharmaceutically acceptable salts thereof. In some examples, the carborane cluster can include a heteroatom.

In some embodiments, the compound can be a carborane analog, such as a dicarba-closo-dodecaborane analog of, for example, the compounds described in WO 2017/049307 to Tjarks et al. The compounds include a spacer group which replaces the carborane moiety in the compounds therein. The resulting compounds can exhibit similar biological activity to the compounds described in WO 2017/049307.

For example, provided herein are compounds defined by Formula XIV, or a pharmaceutically acceptable salt thereof.

A-Q-R¹ Formula XIV
wherein A is a substituted or unsubstituted aryl ring or a substituted or unsubstituted heteroaryl ring; Q is a spacer group chosen from one of the following:

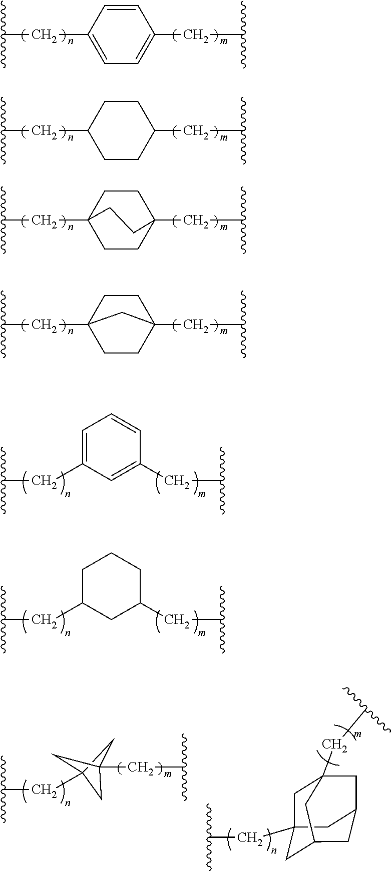

where m and n are each individually 0, 1, 2, or 3; R¹ is substituted or unsubstituted C₄-C₂₀ alkyl, substituted or unsubstituted C₄-C₂₀ heteroalkyl, substituted or unsubstituted C₂-C₂₀ alkenyl, substituted or unsubstituted C₂-C₂₀ alkynyl, substituted or unsubstituted C₃-C₂₀ alkylaryl, substituted or unsubstituted C₃-C₂₀ alkylheteroaryl, substituted or unsubstituted C₄-C₂₀ alkylcycloalkyl, substituted or unsubstituted C₄-C₂m alkylheterocycloalkyl, substituted or unsubstituted C₁-C₂₀ acyl, C₁-C₂₀ acyl, —C(O)NR³R⁴, or NR³R⁴; and R³ and R⁴ are independently selected from substituted or unsubstituted C₁-C₂₀ alkyl, substituted or unsubstituted C₁-C₂₀ heteroalkyl, substituted or unsubstituted C₂-C₂₀ alkenyl, substituted or unsubstituted C₂-C₂₀ alkynyl, substituted or unsubstituted C₂-C₂₀ alkylaryl, or substituted or unsubstituted C₄-C₂₀ alkylcycloalkyl.

In certain embodiments, Q can be chosen from one of the following:

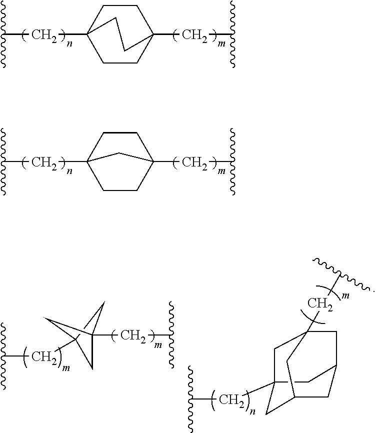

In some embodiments, A can comprise a substituted or unsubstituted aryl ring (e.g., a substituted or unsubstituted phenyl ring). In some embodiments, A can be a five-membered substituted or unsubstituted heteroaryl ring. For example, A can comprise a thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, or 1,3,4-oxadiazolyl ring. In some embodiments, A can be a six-membered substituted or unsubstituted heteroaryl ring. For example, A can comprise a pyridyl, pyrazinyl, pyrimidinyl, triazinyl, or pyridazinyl ring.

In some embodiments, A is

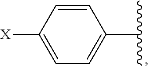

wherein X is OH, NHR², SH, or S(O)(O)NHR² and R² is H, OH, halogen, or substituted or unsubstituted C₁-C₄ alkyl. In some of these embodiments, X is OH.

In some embodiments, A is

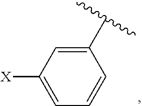

wherein X is OH, NHR², SH, or S(O)(O)NHR²; and R² is H, OH, halogen, or substituted or unsubstituted C₁-C₄ alkyl. In some of these embodiments, X is OH.

In some embodiments, A is

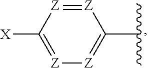

wherein Z is, individually for each occurrence, N or CH, with the proviso that at least one of Z is N; X is OH, NHR², SH, or S(O)(O)NHR², and R² is H, OH, halogen, or substituted or unsubstituted C₁-C₄ alkyl. In some of these embodiments, A can be one of the following:

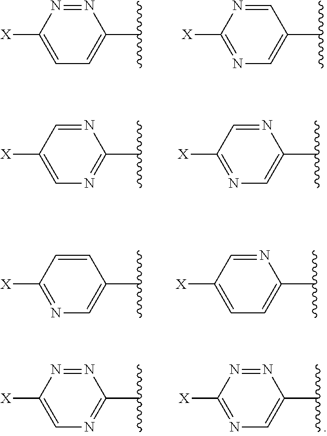

In some of these embodiments, X is OH.

In some embodiments, A is

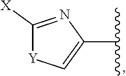

wherein Y is S or O; X is OH, NHR², SH, or S(O)(O)NHR²; and R² is H, OH, halogen, or substituted or unsubstituted C₁-C₄ alkyl. In some of these embodiments, X is OH.

In some embodiments, A is

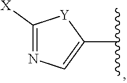

wherein Y is S or O; X is OH, NHR², SH, or S(O)(O)NHR²; and R² is H, OH, halogen, or substituted or unsubstituted C₁-C₄ alkyl. In some of these embodiments, X is OH.

In some embodiments, A is

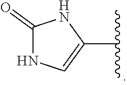 [SMILES: *c1c[nH]c(=O)[nH]1]

In some of the embodiments above, R¹ can be a substituted or unsubstituted C₆-C₁₀ alkyl (e.g., a C₆-C₁₀ hydroxyalkyl).

In some of the embodiments above, R¹ can be a substituted or unsubstituted C₃-C₁₆ alkylaryl (e.g., a C₃-C₁₆ hydroxyalkylaryl).

In some of the embodiments above, R¹ can be a substituted or unsubstituted C₅-C₂₀ alkylaryl (e.g., a C₈-C₂₀ hydroxyalkylaryl).

In some of the embodiments above, R¹ can be a substituted or unsubstituted C₅-C₁₀ acyl.

In some of the embodiments above, R¹ can be a substituted or unsubstituted branched C₄-C₁₀ alkyl (e.g., a branched C₄-C₁₀ hydroxyalkyl).

In some embodiments, R¹ can comprise one of the following:

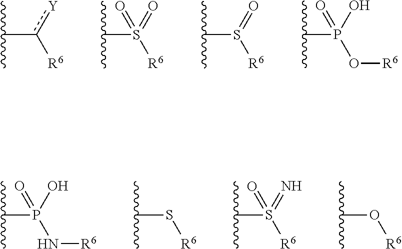

wherein the dotted line to Y indicates that the bond can be a single bond or a double bond, as valence permits; Y, when present, is O, halogen, OR^{2′}, NHR², SH, or S(O)(O)NHR²; R⁶ is substituted or unsubstituted C₁-C₁₉ alkyl, substituted or unsubstituted C₂-C₁₉ alkenyl, substituted or unsubstituted C₂-C₁₉ alkynyl, substituted or unsubstituted C₂-C₁₉ alkylaryl, substituted or unsubstituted C₂-C₁₉ alkylheteroaryl, substituted or unsubstituted C₄-C₁₉ alkylcycloalkyl, substituted or unsubstituted C₄-C₁₉ alkylheterocycloalkyl, and substituted or unsubstituted C₂-C₂₀ heteroalkyl, or NR³R⁴; R² is H, OH, halogen, or substituted or unsubstituted C₁-C₄ alkyl; R^{2′} is H or substituted or unsubstituted C₁-C₄ alkyl; and R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, and substituted or unsubstituted C₂-C₂₀ heteroalkyl.

In some embodiments, A can comprise a substituted or unsubstituted aryl ring (e.g., a substituted or unsubstituted phenyl ring). In some embodiments, A can be a five-membered substituted or unsubstituted heteroaryl ring. For example, A can comprise a thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, or 1,3,4-oxadiazolyl ring. In some embodiments, A can be a six-membered substituted or unsubstituted heteroaryl ring. For example, A can comprise a pyridyl, pyrazinyl, pyrimidinyl, triazinyl, or pyridazinyl ring.

In some of these embodiments, Y is OH. In some of these embodiments, Y is F. In some of these embodiments, Y is O.

In some examples, R⁶ can be a substituted or unsubstituted C₃-C₁₀ alkyl, such as a substituted or unsubstituted C₆-C₉ alkyl.

In some examples, R⁶ can be a substituted or unsubstituted C₂-C₁₅ alkylaryl.

in some examples, R⁶ can be a substituted or unsubstituted branched C₂-C₉ alkyl.

In some examples, R⁶ can be a substituted or unsubstituted C₁-C₁₀ heteroalkyl, such as a substituted or unsubstituted C₆-C₉ heteroalkyl.

In some embodiments, the compound can comprise one of the following:

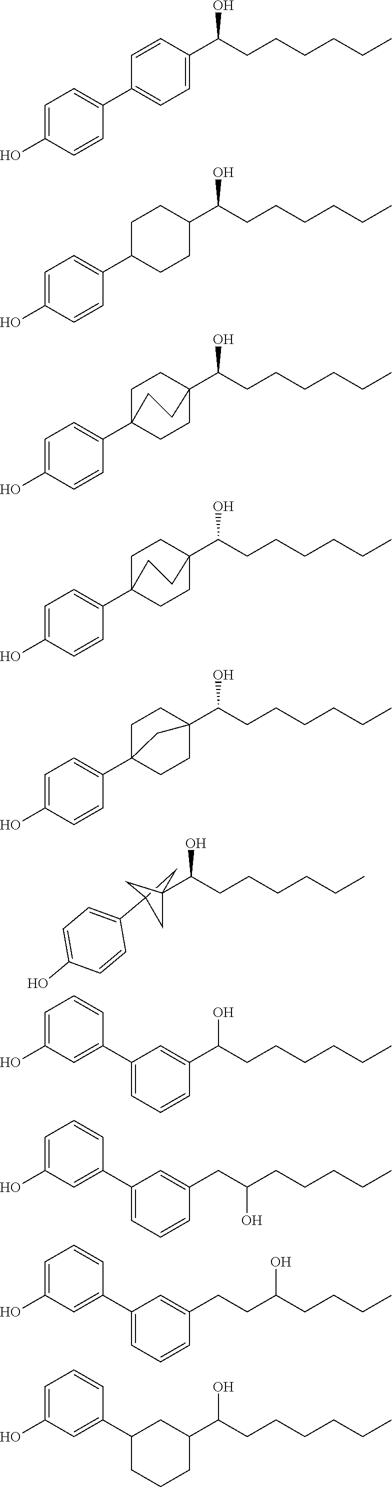

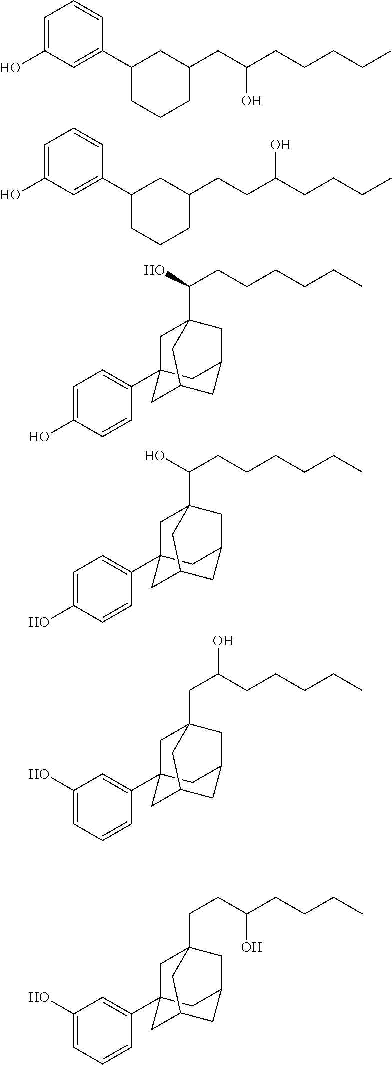

Also disclosed herein are pharmaceutically-acceptable salts and prodrugs of the carboranes and carborane analogs described herein. Pharmaceutically-acceptable salts include salts of the disclosed carboranes and carborane analogs that are prepared with acids or bases, depending on the particular substituents found on the compounds. Under conditions where the carboranes and carborane analogs disclosed herein are sufficiently basic or acidic to form stable nontoxic acid or base salts, administration of the compounds as salts can be appropriate. Examples of pharmaceutically-acceptable base addition salts include sodium, potassium, calcium, ammonium, or magnesium salt. Examples of physiologically-acceptable acid addition salts include hydrochloric, hydrobromic, nitric, phosphoric, carbonic, sulfuric, and organic acids like acetic, propionic, benzoic, succinic, fumaric, mandelic, oxalic, citric, tartaric, malonic, ascorbic, alpha-ketoglutaric, alpha-glycophosphoric, maleic, tosyl acid, methanesulfonic, and the like. Thus, disclosed herein are the hydrochloride, nitrate, phosphate, carbonate, bicarbonate, sulfate, acetate, propionate, benzoate, succinate, fumarate, mandelate, oxalate, citrate, tartarate, malonate, ascorbate, alpha-ketoglutarate, alpha-glycophosphate, maleate, tosylate, and mesylate salts. Pharmaceutically acceptable salts of a compound can be obtained using standard procedures well known in the art, for example, by reacting a sufficiently basic compound such as an amine with a suitable acid affording a physiologically acceptable anion. Alkali metal (for example, sodium, potassium, or lithium) or alkaline earth metal (for example calcium) salts of carboxylic acids can also be made.

In some examples, the carboranes and carborane analogs can have an EC₅₀ of 800 nM or less at estrogen receptor beta (ERβ) (e.g., 700 nM or less, 600 nM or less, 500 nM or less, 400 nM or less, 300 nM or less, 200 nM or less, 100 nM or less, 90 nM or less, 80 nM or less, 70 nM or less, 60 nM or less, 50 nM or less, 40 nM or less, 30 nM or less, 20 nM or less, 10 nM or less, 9 nM or less, 8 nM or less, 7 nM or less, 6 nM or less, 5 nM or less, 4.5 nM or less, 4 nM or less, 3.5 nM or less, 3 nM or less, 2.5 nM or less, 2 nM or less, 1.5 nM or less, 1 nM or less, 0.9 nM or less, 0.8 nM or less, 0.7 nM or less, 0.6 nM or less, 0.5 nM or less, 0.4 nM or less, 0.3 nM or less, 0.2 nM or less, or 0.1 nM or less).

In some examples, the carboranes and carborane analogs can have an EC₅₀ of 1 pM or more at ERβ (e.g., 0.1 nM or more, 0.2 nM or more, 0.3 nM or more, 0.4 nM or more, 0.5 nM or more, 0.6 nM or more, 0.7 nM or more, 0.8 nM or more, 0.9 nM or more, 1 nM or more, 1.5 nM or more, 2 nM or more, 2.5 nM or more, 3 nM or more, 3.5 nM or more, 4 nM or more, 4.5 nM or more, 5 nM or more, 6 nM or more, 7 nM or more, 8 nM or more, 9 nM or more, 10 nM or more, 20 nM or more, 30 nM or more, 40 nM or more, 50 nM or more, 60 nM or more, 70 nM or more, 80 nM or more, 90 nM or more, 100 nM or more, 200 nM or more, 300 nM or more, 400 nM or more, 500 nM or more, 600 nM or more, or 700 nM or more).

The EC₅₀ of the carboranes and carborane analogs at ERβ can range from any of the minimum values described above to any of the maximum values described above. For example, the carboranes and carborane analogs can have an EC₅₀ of from 1 pM to 800 nM at ERs (e.g., from 1 pM to 400 nM, from 400 nM to 800 nM, from 1 pM to 300 nM, from 1 pM to 200 nM, from 1 pM to 100 nM, from 1 pM to 50 nM, from 1 pM to 20 nM, from 1 pM to 10 nM, from 1 pM to 6 nM, from 1 pM to 5 nM, from 1 pM to 2 nM, from 1 pM to 1 nM, from 1 pM to 0.7 nM, from 1 pM to 0.5 nM, from 1 pM to 0.2 pM, or from 1 pM to 0.1 nM).

In some examples, the carboranes and carborane analogs are selective ERβ agonist. In some examples, a selective ERβ agonist is a compound that has a lower EC₅₀ at ERβ than at estrogen receptor α (ERα). The selectivity of the compounds can, in some examples, be expressed as an ERβ-to-ERα agonist ratio, which is the EC₅₀ of the compound at ERα divided by the EC₅₀ of the compound at ERβ. In some examples, the compounds can have an ERβ-to-ERα agonist ratio of 8 or more (e.g., 10 or more, 20 or more, 30 or more, 40 or more, 50 or more, 60 or more, 70 or more, 80 or more, 90 or more, 100 or more, 150 or more, 200 or more, 250 or more, 300 or more, 350 or more, 400 or more, 450 or more, 500 or more, 600 or more, 700 or more, 800 or more, 900 or more, 1000 or more, 1100 or more, 1200 or more, 1300 or more, 1400 or more, 1500 or more, 2000 or more, 2500 or more).

In some examples, the carboranes and carborane analogs can have an ERβ-to-ERα agonist ratio of 3000 or less (e.g., 2500 or less, 2000 or less, 1500 or less, 1400 or less, 1300 or less, 1200 or less, 1100 or less, 1000 or less, 900 or less, 800 or less, 700 or less, 600 or less, 500 or less, 450 or less, 400 or less, 350 or less, 300 or less, 250 or less, 200 or less, 150 or less, 100 or less, 90 or less, 80 or less, 70 or less, 60 or less, 50 or less, 40 or less, 30 or less, 20 or less, or 10 or less).

The ERβ-to-ERα agonist ratio of the carboranes and carborane analogs at ERβ can range from any of the minimum values described above to any of the maximum values described above. For example, the carboranes and carborane analogs can have an ERβ-to-ERα agonist ratio of from 8 to 3000 (e.g., from 8 to 1500, from 1500 to 3000, from 400 to 3000, from 500 to 3000, from 600 to 3000, from 700 to 3000, from 800 to 3000, from 900 to 3000, from 1000 to 3000, or from 2000 to 3000).

### Methods of Making

The compounds described herein can be prepared in a variety of ways known to one skilled in the art of organic synthesis or variations thereon as appreciated by those skilled in the art. The compounds described herein can be prepared from readily available starting materials. Optimum reaction conditions can vary with the particular reactants or solvents used, but such conditions can be determined by one skilled in the art.

Variations on the compounds described herein include the addition, subtraction, or movement of the various constituents as described for each compound. Similarly, when one or more chiral centers are present in a molecule, the chirality of the molecule can be changed. Additionally, compound synthesis can involve the protection and deprotection of various chemical groups. The use of protection and deprotection, and the selection of appropriate protecting groups can be determined by one skilled in the art. The chemistry of protecting groups can be found, for example, in Wuts and Greene, Protective Groups in Organic Synthesis, 4th Ed., Wiley & Sons, 2006, which is incorporated herein by reference in its entirety.

The starting materials and reagents used in preparing the disclosed compounds and compositions are either available from commercial suppliers such as Katchem (Prague, Czech Republic), Aldrich Chemical Co., (Milwaukee, WI), Acros Organics (Morris Plains, NJ), Fisher Scientific (Pittsburgh, PA), Sigma (St. Louis, MO), Pfizer (New York, NY), GlaxoSmithKline (Raleigh, NC), Merck (Whitehouse Station, NJ), Johnson & Johnson (New Brunswick, NJ), Aventis (Bridgewater, NJ), AstraZeneca (Wilmington, DE), Novartis (Basel, Switzerland), Wyeth (Madison, NJ), Bristol-Myers-Squibb (New York, NY), Roche (Basel, Switzerland), Lilly (Indianapolis, IN), Abbott (Abbott Park, IL), Schering Plough (Kenilworth, NJ), or Boehringer Ingelheim (Ingelheim, Germany), or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989). Other materials, such as the pharmaceutical excipients disclosed herein can be obtained from commercial sources.

Reactions to produce the compounds described herein can be carried out in solvents, which can be selected by one of skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high-performance liquid chromatography (HPLC) or thin layer chromatography.

Example methods of preparing carboranes and carborane analogs are described, for example, in U.S. Pat. No. 6,838,574 to Endo, U.S. Patent Application Publication No. 2018/0264017 to Tjarks et al., and PCT/US2019/064228 to Coss et al., and PCT/US2021/021909 to Bansal et al., each of which is hereby incorporated by reference in its entirety.

### Methods of Use

The carboranes and carborane analogs described herein can be administered to a subject in need thereof to treat lupus, prevent lupus development in a predisposed subject, and/or prevent lupus flare.

Accordingly, in one aspect, a method of reducing a lupus associated symptom in a subject is disclosed. These methods can comprise administering a carborane or carborane analog described herein (e.g., as a single agent or in combination with another agent or therapeutic modality) in an amount sufficient to decrease or inhibit lupus (e.g., to decrease lupus associated symptoms or lupus associated biological processes). In one embodiment, the method is carried out in vivo, for example, in a mammalian subject, e.g., an animal model or as part of therapeutic protocol.

Also disclosed herein are methods of treating or preventing lupus in a subject. The methods include administering a carborane or carborane analog (e.g., as a single agent or in combination with another agent or therapeutic modality), to a subject in need thereof, in an amount sufficient to decrease lupus or a lupus associated symptom in the subject.

As used herein, a “symptom” associated with lupus includes, for example, any signs or symptoms of lupus as disclosed herein or as known in the art (e.g., joint pain and stiffness; muscle aches, pains, or weakness, fever; malaise; cutaneous manifestations (e.g., butterfly-shaped rash across the nose and cheeks; other skin rashes); unusual weight loss or weight gain; anemia; neurological or neuropsychiatric manifestations (e.g., trouble thinking, memory problems, confusion, depression, headache, seizures, strokes); kidney problems (e.g., nephritis, e.g., glomerulonephritis); chest pain; sun or light sensitivity; hair loss; Raynaud's phenomenon; vascular lesions or other vascular manifestations (e.g., Raynaud's phenomenon, nail fold telangiectasia and infarct, splinter hemorrhages, chilblain LE, acquired C₁ esterase deficiency, vasculitis, urticarial vasculitis, purpura, thrombophlebitis, livedo reticularis, antiphospholipid syndrome, Degos syndrome and calcinosis)) as well as biological concomitants of lupus as disclosed herein or as known in the art (e.g., immune complexes, elevated levels of cytokines (e.g., interferons (e.g., Type I interferons, e.g., IFN-α and/or IFN-s); interleukins (e.g., IL-6, IL-8, IL-1, and IL-18) and TNF-α), elevated levels of anti-dsDNA autoantibodies, overexpression of IFN-α and/or IFN-β inducible genes, elevated levels of IP-10, elevated levels of sCD40L, reduced levels of C3-derived C₃b, reduced peripheral iNKT cell frequencies, defective B cell-mediated stimulation of iNKT cells, altered CD1d expression on B cells, reduced numbers of natural regulatory T cells (Treg)). Symptoms can be assessed using assays and scales (e.g., Systemic Lupus Activity Measure-Revised (SLAM-R)) disclosed and/or exemplified herein and/or as known in the art. See, e.g., Willis, R. et al. (2012) Lupus, 21: 830-835; Yao, Y. et al. (2009) Arthritis & Rheumatism, 60(6): 1785-1796; Le Buanec, H. (2011) PNAS. 108(47):18995-19000; Bosma, A. et al. (2012) Immunity, 36, 477-490; Wenzel, J. et al. (2010) Lupus, 19: 1020-1028.

In some embodiments, the symptom is nephritis (e.g., glomerulonephritis), proteinuria, or spleen inflammation. In certain embodiments, the symptom is glomerulonephritis. In some embodiments, the symptom is an immune complex. In some embodiments, the symptom is a cutaneous manifestation of lupus. In some embodiments, the symptom is overexpression of a cytokine, such as IL-1, IL-2, IL-4, IL-6, IL-8, IL-10, IL-12p70, IL-13, IFNγ, or TNFα.

As used herein, to “decrease,” “ameliorate,” “reduce,” “inhibit,” “treat” (or the like) lupus or a symptom associated with lupus includes reducing (or preventing an increase in) the severity and/or frequency of one or more symptoms of lupus, as well as preventing lupus and/or one or more symptoms of lupus (e.g., by reducing (or preventing an increase in) the severity and/or frequency of flares of symptoms). In the context of biological molecules, to “decrease”, “ameliorate,” “reduce,” “inhibit,” or the like, includes decreasing the level (e.g., the level, e.g., of mRNA or protein, that can be measured in a biological sample) or the activity (e.g., the function) of the molecule.

In some embodiments, the symptom is reduced by at least 10%, at least 20%, at least 30%, at least 40%, at least 50%, at least 60%, at least 70%, at least 80%, at least 90%, or at least 95% relative to a control level. The control level includes any appropriate control as known in the art. For example, the control level can be the pre-treatment level in the sample or subject treated, or it can be the level in a control population (e.g., the level in subjects who do not have lupus or the level in samples derived from subjects who do not have lupus). In some embodiments, the decrease is statistically significant, for example, as assessed using an appropriate parametric or non-parametric statistical comparison.

As used herein, “lupus” refers to all types and manifestations of lupus. Manifestations of lupus include, without limitation, systemic lupus erythematosus; lupus nephritis; cutaneous manifestations (e.g., manifestations seen in cutaneous lupus erythematosus, e.g., a skin lesion or rash); CNS lupus; cardiovascular, pulmonary, hepatic, haematological, gastrointestinal and musculoskeletal manifestations; neonatal lupus erythematosus; childhood systemic lupus erythematosus; drug-induced lupus erythematosus; anti-phospholipid syndrome; and complement deficiency syndromes resulting in lupus manifestations.

In some embodiments, the lupus is selected from systemic lupus erythematosus (SLE), cutaneous lupus erythematosus (CLE), drug-induced lupus, and neonatal lupus.

In some embodiments, the lupus is a CLE, e.g., acute cutaneous lupus erythematosus (ACLE), subacute cutaneous lupus erythematosus (SCLE), intermittent cutaneous lupus erythematosus (also known as lupus erythematosus tumidus (LET)), or chronic cutaneous lupus. In some embodiments, the intermittent CLE is selected from chronic discloid lupus erythematosus (CDLE) and lupus erythematosus profundus (LEP) (also known as lupus erythematosus panniculitis). Types, symptoms, and pathogenesis of CLE are described in Wenzel, J. et al. (2010), Lupus, 19, 1020-1028.

In some embodiments, the subject is a mammal. In some embodiments, the subject is a human. In certain embodiments, the subject is an animal model of lupus, a human with lupus, or a subject (e.g., a human) at risk for developing lupus. In some embodiments, the subject is a human who has a family history of lupus, who carries a gene associated with lupus, who is positive for a biomarker associated with lupus, or a combination thereof. In some embodiments, the subject has been diagnosed with lupus. In some embodiments, the subject has one or more signs or symptoms associated with lupus. In some embodiments, the subject is at risk for developing lupus (e.g., the subject carries a gene that, individually, or in combination with other genes or environmental factors, is associated with development of lupus).

In some embodiments, the subject exhibits elevated levels of antinuclear antibodies (e.g., anti-Smith antibodies, anti-double stranded DNA (dsDNA) antibodies, anti-U1 RNP, SS-a (or anti-Ro), SS-b (or anti-La)), antiphospholipid antibodies, anti-ss DNA antibodies, anti-histone antibodies, or anticardiolipin antibodies. In some embodiments, the subject exhibits elevated levels of anti-dsDNA antibodies. In some embodiments, the subject exhibits elevated levels of anti-Sm antibodies.

In some embodiments, the subject exhibits in the peripheral blood lower levels of complement C3, C4, CH50 or complement receptor 1 on erythrocytes (ECR1). In some embodiments, the subject exhibits elevated levels of complement C4d on erythrocytes (EC4d), platelets (PC4d), and B cells (BC4d).

In some embodiments, the subject exhibits autoantibodies against one or more antigens that are known to be associated with lupus or with lupus subtypes. In some embodiments, the subject exhibits autoantibodies against Sm/anti-RNP or Ro/La autoantigens.

The levels of antibodies associated with lupus can be assessed using methods known in the art, e.g., indirect immunofluorescence, immunodiffusion, immunoprecipitation or immunoblotting. In some embodiments, the methods disclosed herein reduce or prevent an increase in the levels of one or more of the foregoing antibodies.

In some embodiments, the subject exhibits elevated levels of a cytokine, such as IL-1, IL-2, IL-4, IL-6, IL-8, IL-10, IL-12p70, IL-13, IFNγ, or TNFα.

In some embodiments, the subject has a mutation (e.g., an SNP) in a gene associated with lupus. In one embodiment, the gene is selected from STAT4, IRF5, BANK1, ITGAM, PD1, FAM167A-BLK, IRF5-TNP03, KIAA1542, TNFAIP3, XKR6, 1q25.1, PXK, ATG5, ICA1, XKR6, LYN and SCUB2 or a combination thereof. See, e.g., Jarvinen, T. M. et al. (2012) Rheumatology, 51:87-92. In one embodiment, the subject is a non-European (e.g., an African American) who carries the disease risk allele of UBE2L3. See, e.g., Agik, S. et al. Journal of Rheumatology (2012), 39(1): 73-78.

In some embodiments, the subject carries the DR3 and DQ2 variants, or the DR2 and DQ6 variants of HLA class II genes.

In some embodiments, the subject has a deficiency in one or more complement proteins, e.g., a deficiency of a complement protein coded by the C4A or C2 genes on chromosome 6, or the C1r and C1s genes on chromosome 12.

In some embodiments, the subject is a subject who is suffering from an active lupus episode. In some embodiments, the subject has inactive lupus.

In some embodiments, the subject is an animal model of lupus Animal models of lupus are known in the art and include, e.g., the murine NZB/W F1 lupus model, which has many features of human lupus and is characterized by elevated levels of anti-nuclear and anti-dsDNA autoantibodies; an important role for plasmacytoid dendritic cells and IFN-α; T-cell, B-cell, macrophage involvement; pheymolytic anemia; progressive immune complex glomerlonephritis; proteinurea; severity and incidence more pronounced in females; and decreased survival.

Other animal models include, e.g., the MRL/lpr, NZB/W, and BSXB mouse strain, as well as transgenic forms of these. See, e.g., Ghoreishi, M. and Dutz, J. P. (2009) Lupus. 19: 1029-1035; Ohl, K. and Tenbrock, K. (2011) Journal of Biomedicine and Biotechnology, Article ID 432595 (14 pages). In some embodiments, the animal model is an animal (e.g., a mouse) that has been injected with Trypanosoma equiperdum (TE). In some embodiments, the animal model is an animal (e.g., a mouse) that has been treated witholl-like receptor 7 agonists. In some embodiments, the animal model is an animal (e.g., a mouse) that has been humanized and lupus induced using a compound know to cause lupus in mice. See, e.g., Xia, Y. et al. (2011) Rheumatology, 50:2187-2196. Further animal models are described, e.g., in Pau, E. et al. (2012) PLoS ONE, 7(5):e36761 (15 pages); Mustafa, A. et al. (2011) Toxicology, 290:156-168; Ichikawa, H. T. et al. (2012) Arthritis and Rheumatism, 62(2): 493-503: Ouyang, S. et al. (2012). J Mol Med, DOI 10.1007/s00109-012-0866-3 (10 pages); Rankin, A. L. et al. (2012) Journal of Immunology, 188:1656-1667.

In some embodiments, the subject is female. In some embodiments, the subject is a female between the ages of 15 and 45. In some embodiments, the subject is not white (e.g., the subject is African-American, of African ancestry, of Latin American ancestry, of Asian ancestry, or of Native American ancestry). In some embodiments, the subject is a female who is not white. In some embodiments, the subject is of African ancestry. See, e.g., Ko, K. et al. (2012) Journal of Rheumatology, 39(1):73-78. In some embodiments, the subject is a female of African ancestry.

In some embodiments, the methods disclosed herein result in inhibition of immune complexes, cytokines (e.g., interferons (e.g., Type I interferons, e.g., IFN-α and/or IFN-s); interleukins (e.g., IL-6, IL-8, and IL-1) and TNF-α), anti-dsDNA autoantibodies, IFN-α and/or IFN-β inducible genes, IP-10, or sCD40L.

some embodiments, the methods disclosed herein reduce nephritis (e.g., glomerulonephritis).

### Compositions, Formulations and Methods of Administration

In vivo application of the disclosed compounds, and compositions containing them, can be accomplished by any suitable method and technique presently or prospectively known to those skilled in the art. For example, the disclosed compounds can be formulated in a physiologically- or pharmaceutically-acceptable form and administered by any suitable route known in the art including, for example, oral, nasal, rectal, topical, and parenteral routes of administration. As used herein, the term parenteral includes subcutaneous, intradermal, intravenous, intramuscular, intraperitoneal, and intrasternal administration, such as by injection. Administration of the disclosed compounds or compositions can be a single administration, or at continuous or distinct intervals as can be readily determined by a person skilled in the art.

The compounds disclosed herein, and compositions comprising them, can also be administered utilizing liposome technology, slow release capsules, implantable pumps, and biodegradable containers. These delivery methods can, advantageously, provide a uniform dosage over an extended period of time. The compounds can also be administered in their salt derivative forms or crystalline forms.

The compounds disclosed herein can be formulated according to known methods for preparing pharmaceutically acceptable compositions. Formulations are described in detail in a number of sources which are well known and readily available to those skilled in the art. For example, Remington's Pharmaceutical Science by E. W. Martin (1995) describes formulations that can be used in connection with the disclosed methods. In general, the compounds disclosed herein can be formulated such that a therapeutically effective amount of the compound is combined with a suitable excipient in order to facilitate effective administration of the compound. The compositions used can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically-acceptable carriers and diluents which are known to those skilled in the art. Examples of carriers or diluents for use with the compounds include ethanol, dimethyl sulfoxide, glycerol, alumina, starch, saline, and equivalent carriers and diluents. To provide for the administration of such dosages for the desired therapeutic treatment, compositions disclosed herein can advantageously comprise between about 0.1% and 100% by weight of the total of one or more of the subject compounds based on the weight of the total composition including carrier or diluent.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the excipients particularly mentioned above, the compositions disclosed herein can include other agents conventional in the art having regard to the type of formulation in question.

Compounds disclosed herein, and compositions comprising them, can be delivered to a cell either through direct contact with the cell or via a carrier means. Carrier means for delivering compounds and compositions to cells are known in the art and include, for example, encapsulating the composition in a liposome moiety. Another means for delivery of compounds and compositions disclosed herein to a cell comprises attaching the compounds to a protein or nucleic acid that is targeted for delivery to the target cell. U.S. Pat. No. 6,960,648 and U.S. Application Publication Nos. 20030032594 and 20020120100 disclose amino acid sequences that can be coupled to another composition and that allows the composition to be translocated across biological membranes. U.S. Application Publication No. 20020035243 also describes compositions for transporting biological moieties across cell membranes for intracellular delivery. Compounds can also be incorporated into polymers, examples of which include poly (D-L lactide-co-glycolide) polymer for intracranial tumors; poly[bis(p-carboxyphenoxy) propane:sebacic acid] in a 20:80 molar ratio (as used in GLIADEL); chondroitin; chitin; and chitosan.

For the treatment of lupus, the compounds disclosed herein can be administered to a patient in need of treatment in combination with one or more additional therapies, e.g., additional therapies for the treatment of lupus or for the treatment of symptoms of lupus. These other substances or treatments can be given at the same as or at different times from the compounds disclosed herein. Some examples of therapies for the treatment of lupus are disclosed, for example, in Chugh, P. K. (2012) European Journal of Internal Medicine, 23, 212-218. In some embodiments, the other therapy is Belimumab (Benlysta). In some embodiments, the other therapy is an anti-interferon therapy, e.g., AGS-009, Rontalizumab (rhuMAb IFNalpha), Vitamin D3, Sifalimumab (MEDI-545), AMG 811, IFNα Kinoid, or CEP33457. In some embodiments, the other therapy is an IFN-α therapy, e.g., AGS-009, Rontalizumab, Vitamin D3, Sifalimumab (MEDI-545) or IFNα Kinoid. In some embodiments, the other therapy is an anti B-cell therapy, e.g., Epratuzumab, LY2127399, Ocrelizumab, Atacicept, A-623, or SBI-087. In some embodiments, the other therapy is an anti T-cell therapy, e.g. AMG557. In some embodiments, the other therapy is an immunomodulatory or immunosuppressant therapy (e.g., laquinimod, rapamycin, cyclophosphamide (Cytoxan), azathioprine (Imuran, Azasan), mycophenolate (Cellcept), leflunomide (Arava) or methotrexate (Trexall)). In some embodiments, the other therapy is an anti-interleukin therapy, e.g., CNTO 136. In some embodiments, the other therapy is Tamibarotene, N-acetylcysteine, or CDP7657. In some embodiments, the other therapy is hydroxychloroquine treatment. See, e.g., Willis, R. et al. (2012) Lupus, 21: 830-835. In some embodiments, the other therapy is a B cell depletion therapy or anti-CD20 therapy (e.g., rituximab). In some embodiments, the other therapy is a vaccine (e.g., an iDC vaccine as described, e.g., in Xia, Y. et al. (2011) Rheumatology, 50:2187-2196, or an IFN-α vaccine, e.g., IFNα Kinoid). In some embodiments, the other therapy is a proteasome inhibitor (e.g., carfilzomib or bortezomib) or an immunoproteasome inhibitor (e.g., ONX 0914). See, e.g., Ichikawa, H. T. et al. (2012) Arthritis and Rheumatism, 62(2): 493-503. In some embodiments, the other therapy inhibits a Toll-like receptor (e.g., hydroxychloroquine, IMO-3100 (inhibits TLR7 and TLR9), or DV1179 (inhibits TLR7 and TLR9)). In some embodiments, the other therapy is a nonsteroidal anti-inflammatory drug (NSAID). In some embodiments, the other therapy is an anti-malarial medication (e.g., hydroxychloroquine). In some embodiments, the other therapy is a corticosteroid.

In certain examples, compounds and compositions disclosed herein can be locally administered at one or more anatomical sites, such as sites of unwanted cell growth (such as a tumor site or benign skin growth, e.g., injected or topically applied to the tumor or skin growth), optionally in combination with a pharmaceutically acceptable carrier such as an inert diluent. Compounds and compositions disclosed herein can be systemically administered, such as intravenously or orally, optionally in combination with a pharmaceutically acceptable carrier such as an inert diluent, or an assimilable edible carrier for oral delivery. They can be enclosed in hard or soft shell gelatin capsules, can be compressed into tablets, or can be incorporated directly with the food of the patient's diet. For oral therapeutic administration, the active compound can be combined with one or more excipients and used in the form of ingestible tablets, buccal tablets, troches, capsules, elixirs, suspensions, syrups, wafers, aerosol sprays, and the like.

The tablets, troches, pills, capsules, and the like can also contain the following: binders such as gum tragacanth, acacia, corn starch or gelatin; diluents such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring can be added. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac, or sugar and the like. A syrup or elixir can contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound can be incorporated into sustained-release preparations and devices.

Compounds and compositions disclosed herein, including pharmaceutically acceptable salts or prodrugs thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the inclusion of agents that delay absorption, for example, aluminum monostearate and gelatin.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, compounds and agents disclosed herein can be applied as a liquid or solid. However, it will generally be desirable to administer them topically to the skin as compositions, in combination with a dermatologically acceptable carrier, which can be a solid or a liquid. Compounds and agents and compositions disclosed herein can be applied topically to a subject's skin to reduce the size (and can include complete removal) of malignant or benign growths, or to treat an infection site. Compounds and agents disclosed herein can be applied directly to the growth or infection site. Preferably, the compounds and agents are applied to the growth or infection site in a formulation such as an ointment, cream, lotion, solution, tincture, or the like.

Useful solid carriers include finely divided solids such as talc, clay, microcrystalline cellulose, silica, alumina and the like. Useful liquid carriers include water, alcohols or glycols or water-alcohol/glycol blends, in which the compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the compounds and agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

Also disclosed are pharmaceutical compositions that comprise a compound disclosed herein in combination with a pharmaceutically acceptable excipient. Pharmaceutical compositions adapted for oral, topical or parenteral administration, comprising an amount of a compound constitute a preferred aspect. The dose administered to a patient, particularly a human, should be sufficient to achieve a therapeutic response in the patient over a reasonable time frame, without lethal toxicity, and preferably causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

The pharmaceutical compositions can comprise a therapeutically effective amount of a carborane or carborane analog described herein (e.g., WT-IV-012) for the treatment of lupus. Optionally, these compositions can further include at least one additional therapeutic as part of the treatment regimen, such as a corticosteroid (e.g., prednisone, prednisolone, methylprednisolone, betamethasone, dexamethasone, triamcinolone, hydrocortisone, or any combination thereof), an NSAID, an antimalarial, an immunomodulating agent, an immunosuppressive agent, an anticoagulant, or a combination thereof.

Also disclosed are kits that comprise a compound disclosed herein in one or more containers. The disclosed kits can optionally include pharmaceutically acceptable carriers and/or diluents. In one embodiment, a kit includes one or more other components, adjuncts, or adjuvants as described herein. In another embodiment, a kit includes one or more anti-cancer agents, such as those agents described herein. In one embodiment, a kit includes instructions or packaging materials that describe how to administer a compound or composition of the kit. Containers of the kit can be of any suitable material, e.g., glass, plastic, metal, etc., and of any suitable size, shape, or configuration. In one embodiment, a compound and/or agent disclosed herein is provided in the kit as a solid, such as a tablet, pill, or powder form. In another embodiment, a compound and/or agent disclosed herein is provided in the kit as a liquid or solution. In one embodiment, the kit comprises an ampoule or syringe containing a compound and/or agent disclosed herein in liquid or solution form.

A number of embodiments of the invention have been described. Nevertheless, it will be understood that various modifications may be made without departing from the spirit and scope of the invention. Accordingly, other embodiments are within the scope of the following claims.

### EXAMPLES

The following examples are set forth to illustrate the methods and results according to the disclosed subject matter. These examples are not intended to be inclusive of all aspects of the subject matter disclosed herein, but rather to illustrate representative methods and results. These examples are not intended to exclude equivalents and variations which are apparent to one skilled in the art.

### Example 1. Evaluation of WT-IV-012 for the Treatment of Lupus

### Materials and Methods

Studies using Humanized Mice: An example carborane and selective ERβ agonist (WT-IV-012, shown below) was evaluated in a mouse model for lupus.

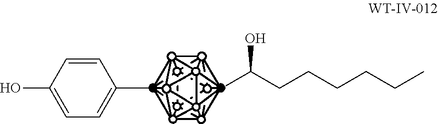

2.5×10⁶-5.0×10⁶ PBMC isolated from patients with active SLE were adoptively transferred into NSG mice and allowed to expand in vivo for one week. The mice were divided into 3 different cohorts receiving either vehicle control, 5 mg/kg prednisone or 50 mg/kg WT-IV-012 via oral gavage on a daily basis for five weeks. Blood samples were taken at baseline, 3 and 5 weeks and serum was purified and analyzed for circulating cytokines using the MSD human V-PLEX Proinflammatory Panel in the Analytical & Development Laboratory, Clinical Research Center at OSU. At 5 weeks, mice were euthanized, kidneys and hearts were harvested and processed for H&E histology at Histowiz (Brooklyn, NY). Subsequently, the digital slide images of the kidneys were analyzed by using Aperio ImageScope v12.1.0.5029 with the Positive Pixel Count v9 algorithm to identify negative pixels. Quantitative data were analyzed with GraphPad Prism v9.0.2 with one-way ANOVA and Tukey's multiple comparison test.

In Vivo Imaging System: The Balb/c-Tg(Rela-luc) mouse line was used to image LPS induced in vivo NFkB activity. Mice were pre-treated with either vehicle control or WT-IV-012 (50 mg/kg) via oral gavage and then injected with LPS in the intraperitoneal cavity. Mice were given 150 mg/kg luciferin [Gold Biotechnology, Inc., St. Louis, MO; and then imaged 4 hours later by using the IVIS Lumina II (Perkin Elmer). Luciferase activity was quantified with Living Image Software v4.4.

### Results and Discussion

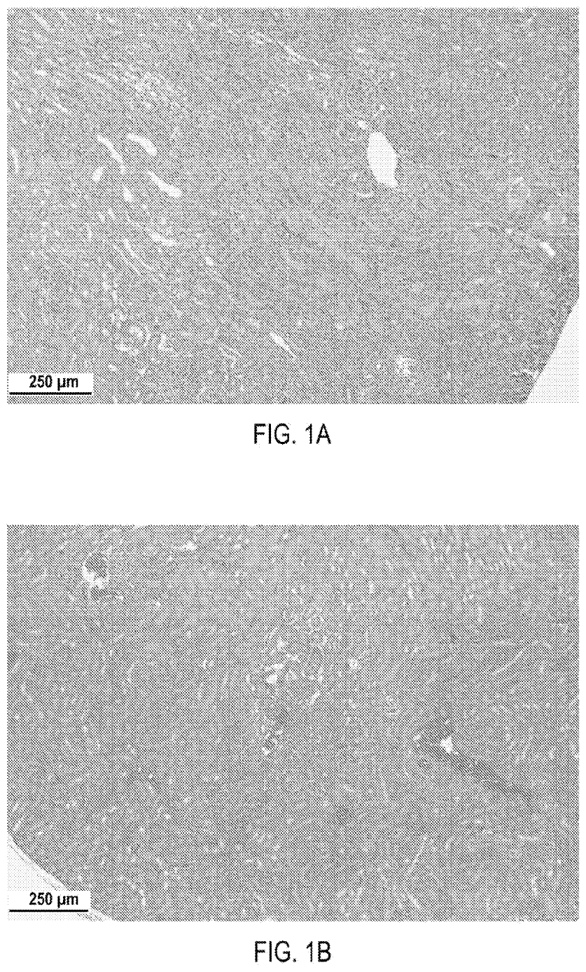

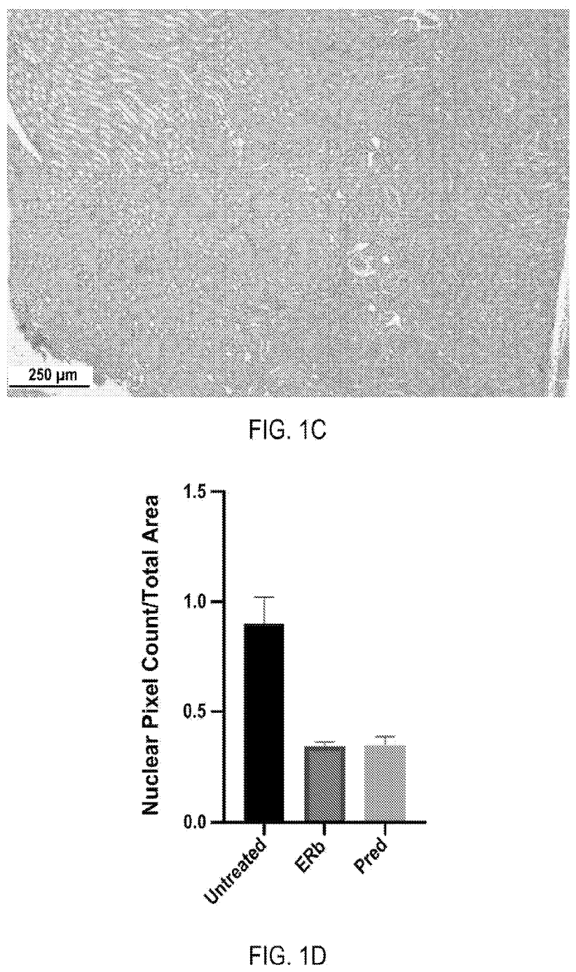

FIGS. 1A-ID illustrate that oral treatment with an example carborane and selective ERβ agonist (WT-IV-012 inhibits kidney nuclear infiltration in SLE Humanized Mice. FIGS. 1A, 1B, and 1C show kidney tissue taken from untreated SLE Humanized Mice (FIG. 1A), SLE Humanized Mice treated with WT-IV-012 (FIG. 1B), and SLE Humanized Mice treated with prednisone (FIG. 1C). FIG. 1D is a plot showing the nuclear pixel count per total area for untreated SLE Humanized Mice, SLE Humanized Mice treated with WT-IV-012, and SLE Humanized Mice treated with prednisone. As shown in FIG. 1D, WT-IV-012 inhibits kidney cellular infiltration to a similar degree as prednisone when tested in a chimeric inflammatory mouse model.

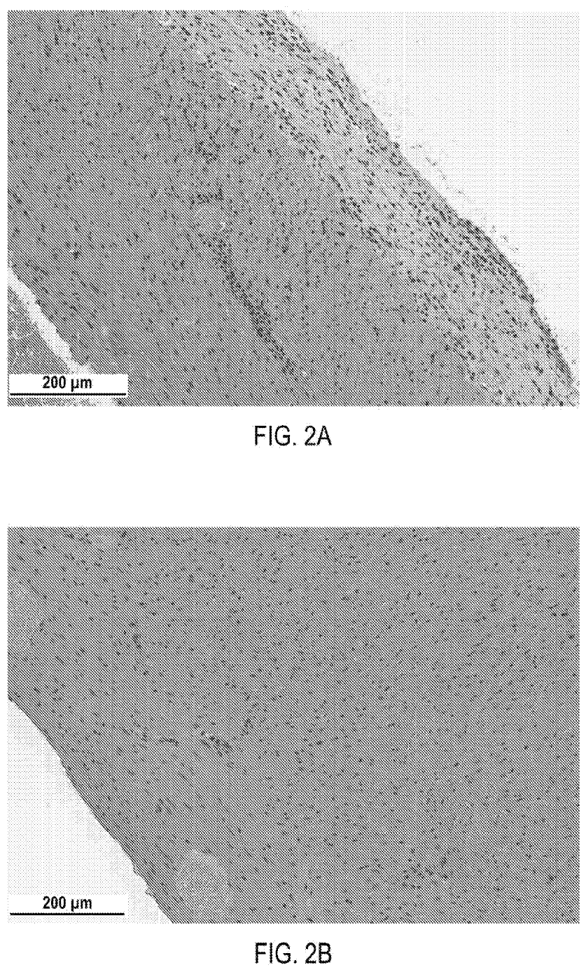

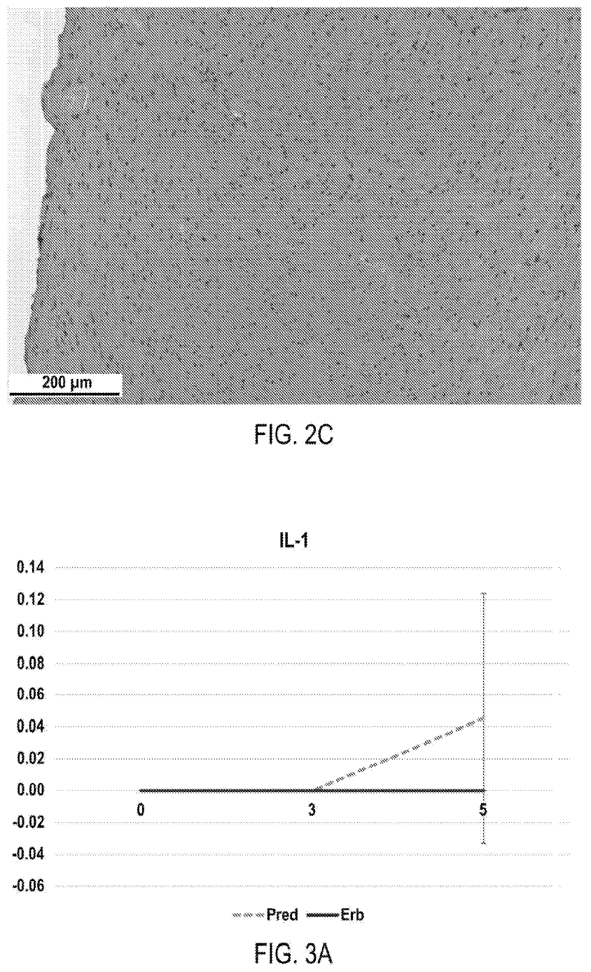

FIGS. 2A-2C illustrate that oral treatment with an example carborane and selective ERβ agonist (WT-IV-012 inhibits heart inflammation in SLE Humanized Mice. FIGS. 2A, 2B, and 2C show heart tissue taken from untreated SLE Humanized Mice (FIG. 2A), SLE Humanized Mice treated with WT-IV-012 (FIG. 2B), and SLE Humanized Mice treated with prednisone (FIG. 2C). As demonstrated by FIGS. 2A-2C, WT-IV-012 inhibits heart inflammation to a similar degree as prednisone when tested in a chimeric inflammatory mouse model.

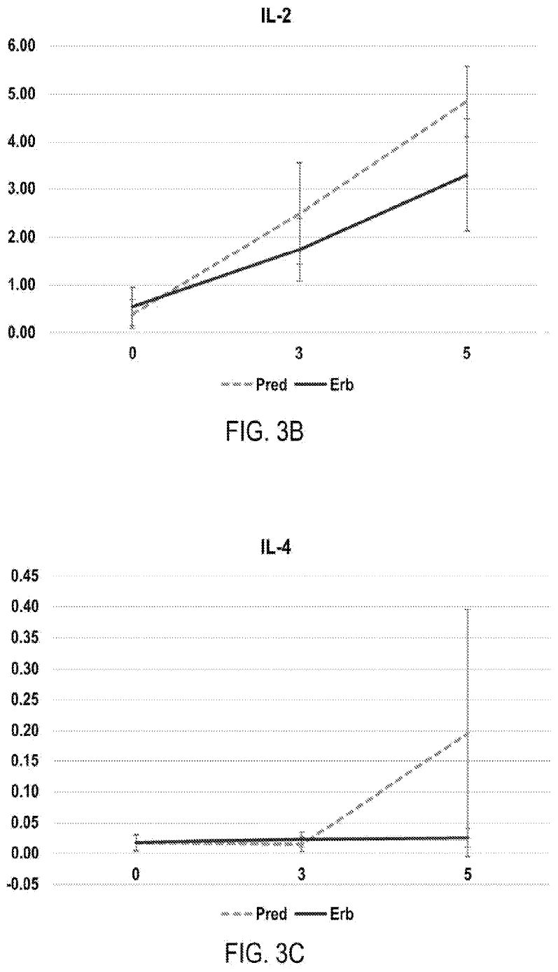

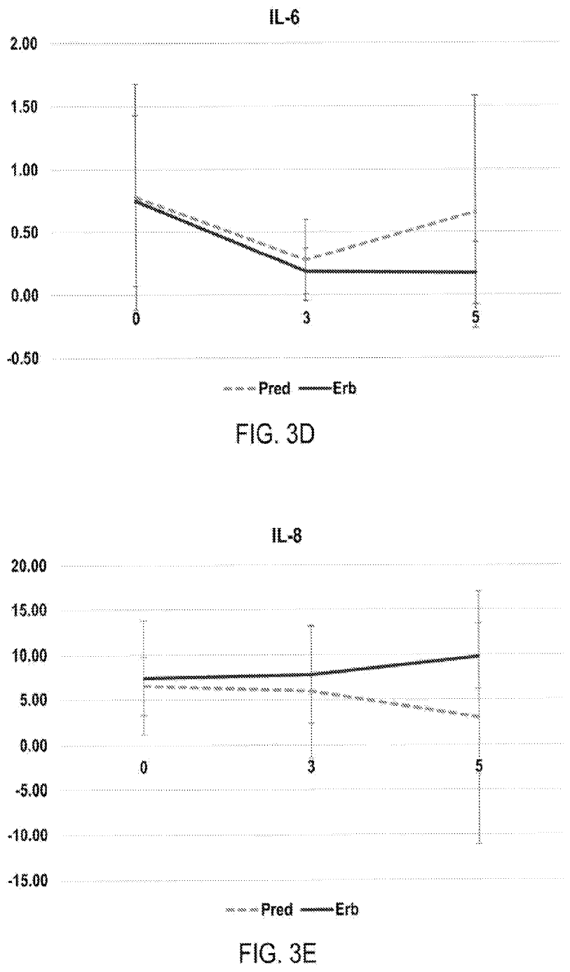

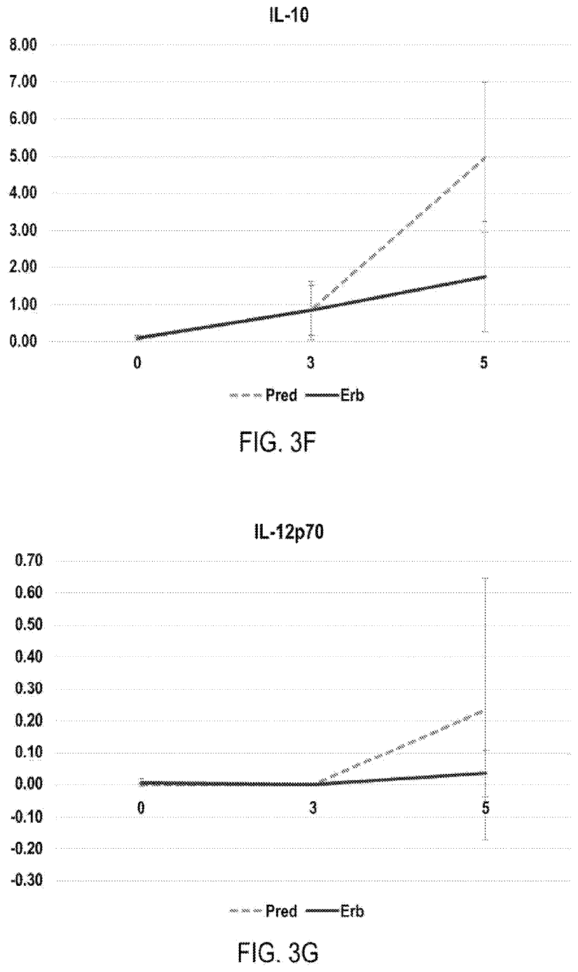

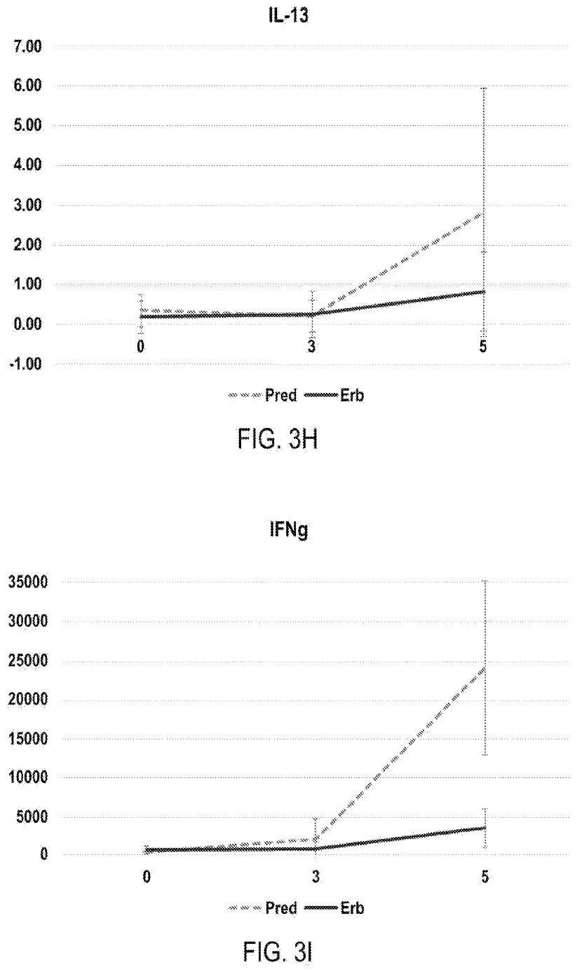

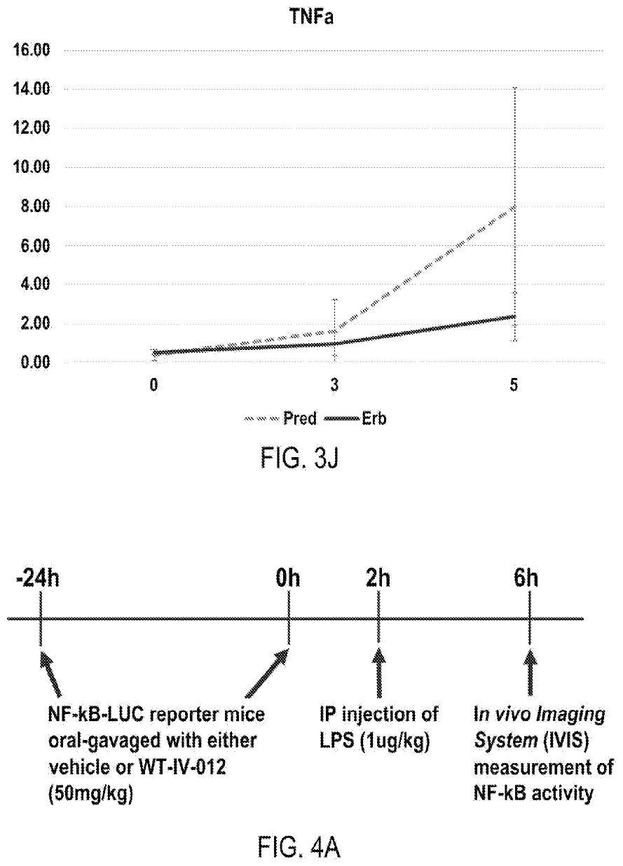

FIGS. 3A-3J show the results an MSD cytokine assay comparing the impact of oral treatment with an example carborane and selective ERβ agonist (WT-IV-012, -Erb) or prednisone (-pred) on the circulating level of ten cytokines that are important in inflammatory responses and immune system regulation. The cytokine levels were assessed at week 0, week 3, and week 5 following administration. WT-IV-012 inhibited, to a greater extent than prednisone, the following cytokines when tested in a chimeric inflammatory mouse model: IL-1 (FIG. 3A), IL-2 (FIG. 3B), IL-4 (FIG. 3C), IL-6 (FIG. 3D), IL-10 (FIG. 3F), IL-12p70 (FIG. 3G), IL-13 (FIG. 3H), IFNγ (FIG. 3I), and TNFα (FIG. 3J). WT-IV-012 appeared to be less effective than prednisone in inhibiting IL-8 (FIG. 3E) when tested in a chimeric inflammatory mouse model.

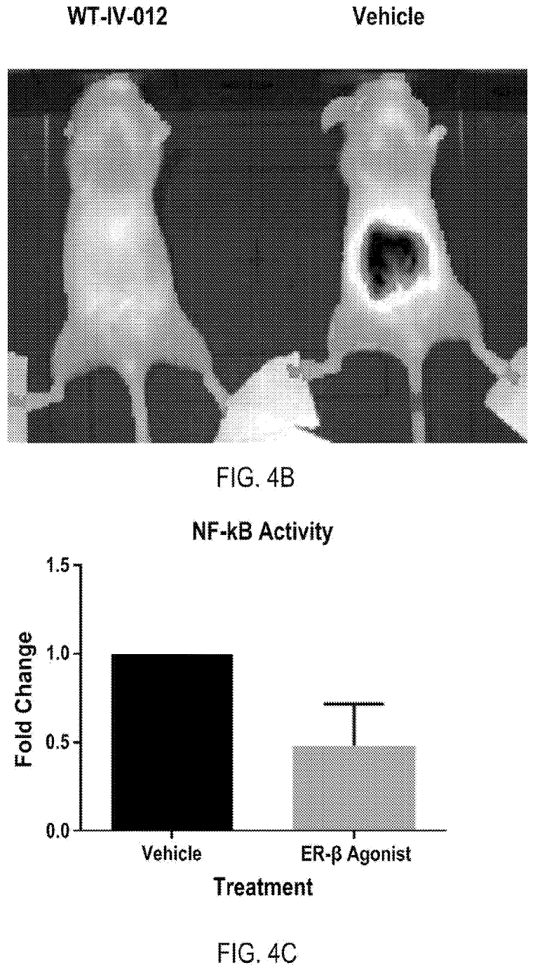

FIG. 4A is a schematic illustration of a study performed to evaluate the effect of an example carborane and selective ERβ agonist (WT-IV-012) on NFKB activity in NF-kB-LUC reporter mice. FIG. 4B is a photograph showing the result of in vivo imaging system (IVIS) measurements of NF-kB activity in NF-kB-LUC reporter mice treated with WT-IV-012 (left) and vehicle alone (right). FIG. 4C is a plot comparing NF-kB activity in NF-kB-LUC reporter mice treated with WT-IV-012 and vehicle alone. These results suggested that WT-IV-012 inhibited LPS-induced NF-KB activation.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method for treating or preventing lupus in a subject in need thereof, the method comprising: administering a therapeutically effective amount of a carborane or carborane analog to the subject.

2. The method of claim 1, wherein the carborane or carborane analog comprises a compound defined by Formula I, or a pharmaceutically acceptable salt thereof 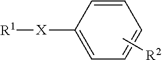
wherein
R¹ represents a dicarba-closo-dodecaboran-yl group which may have one or more substituents selected from the group consisting of an alkyl group, an alkenyl group, a carboxyl group, an alkoxycarbonyl group, an amino group, a hydroxyl group, a hydroxyalkyl group, a mono or di-alkylcarbamoyl-substituted alkyl group, an alkanoyl group, an aryl group, and an aralkyl group, each of which may be substituted or unsubstituted;
R² represents a carboxyl group, an alkoxycarbonyl group, or a hydroxyl group; and
X represents a single bond, or a linking group selected from the group consisting of groups represented by the following formulas: 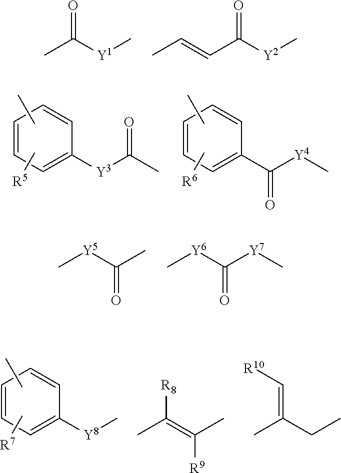
wherein Y¹, Y², Y³, Y⁴, Y⁵, Y⁶, and Y⁷ independently represent an oxygen atom or —N(R³)— wherein R³ represents hydrogen atom or an alkyl group; Y⁸ represents an oxygen atom, —N(R⁴)— wherein R⁴ represents hydrogen atom or an alkyl group, —CO—, —CH₂—, or —C(═CH²)—; R⁵, R⁶, and R⁷ independently represent hydrogen or one or more substituents on the phenyl group; R⁸ represents an alkyl group or an aryl group which may be substituted;
R⁹ represents an alkyl group; and R¹⁰ represents a substituted or unsubstituted aryl group.

3. The method of claim 1, wherein the carborane or carborane analog comprises a compound defined by Formula II, or a pharmaceutically acceptable salt thereof 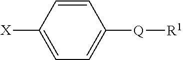
wherein
Q is a substituted or unsubstituted dicarba-closo-dodecaborane cluster, and 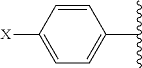
and R¹ are attached to Q in a para configuration;
X is OH, NHR², SH, or S(OXO)NHR²;
R¹ is substituted or unsubstituted C₄-C₂₀ alkyl, substituted or unsubstituted C₂-C₂₀ alkenyl, substituted or unsubstituted C₂-C₂₀ alkynyl, substituted or unsubstituted C₃-C₂₀ alkylaryl, substituted or unsubstituted C₃-C₂₀ alkylheteroaryl, substituted or unsubstituted C₄-C₂₀ alkylcycloalkyl, substituted or unsubstituted C₄-C₂₀ alkylheterocycloalkyl, substituted or unsubstituted C₁-C₂₀ acyl, or NR³R⁴;
R² is H, OH, halogen, or substituted or unsubstituted C₁-C₄ alkyl; and
R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, or substituted or unsubstituted C₁-C₂₀ acyl.

4. The method of claim 1, wherein the carborane or carborane analog comprises a compound defined by Formula III, or a pharmaceutically acceptable salt thereof 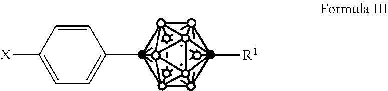
wherein
● is a carbon atom;
◯ is B—H, B-halogen, B-alkyl, B—OH, or B—NH₂;
X is OH, NHR², SH, or S(OXO)NHR²;
R¹ is substituted or unsubstituted C₄-C₂₀ alkyl, substituted or unsubstituted C₂-C₂₀ alkenyl, substituted or unsubstituted C₂-C₂₀ alkynyl, substituted or unsubstituted C₃-C₂₀ alkylaryl, substituted or unsubstituted C₃-C₂₀ alkylheteroaryl, substituted or unsubstituted C₄-C₂₀ alkylcycloalkyl, substituted or unsubstituted C₄-C₂₀ alkylheterocycloalkyl, substituted or unsubstituted C₁-C₂₀ acyl, or NR³R⁴;
R² is H, OH, halogen, or substituted or unsubstituted C₁-C₄ alkyl; and
R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, or substituted or unsubstituted C₁-C₂₀ acyl.

5. The method of claim 1, wherein the carborane or carborane analog comprises a compound defined by Formula IV, or a pharmaceutically acceptable salt thereof 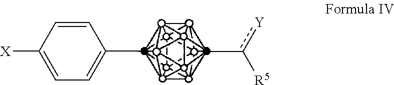
wherein
● is a carbon atom;
◯ is B—H, B-halogen, B-alkyl, B—OH, or B—NH₂;
the dotted line to Y indicates that the bond can be a single bond or a double bond, as valence permits;
X is OH, NHR², SH, or S(OXO)NHR²;
Y is O, OR^{2′}, NHR², SH, or S(OXO)NHR²;
R is substituted or unsubstituted C₂-C₁₉ alkyl, substituted or unsubstituted C₂-C₁₉ alkenyl, substituted or unsubstituted C₂-C₁₉ alkynyl, substituted or unsubstituted C₂-C₁₉ alkylaryl, substituted or unsubstituted C₂-C₁₉ alkylheteroaryl, substituted or unsubstituted C₃-C₁₉ alkylcycloalkyl, substituted or unsubstituted C₃-C₁₉ alkylheterocycloalkyl, or NR³R⁴;
R² is H, OH, halogen, or substituted or unsubstituted C₁-C₄ alkyl;
R^{2′} is H or substituted or unsubstituted C₁-C₄ alkyl; and
R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, or substituted or unsubstituted C₁-C₂₀ acyl.

6. The method of claim 1, wherein the carborane or carborane analog comprises a compound defined by Formula VII, or a pharmaceutically acceptable salt thereof 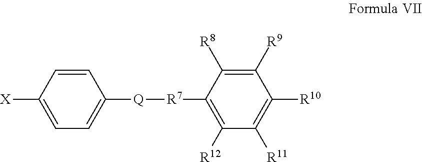
wherein
Q is a substituted or unsubstituted dicarba-closo-dodecaborane cluster, and 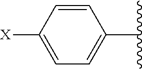
and R⁷ are attached to Q in a para configuration;
X is OH, NHR², SH, or S(OXO)NHR²;
R⁷ is substituted or unsubstituted C₁-C₁₄ alkyl, substituted or unsubstituted C₂-C₁₄ alkenyl, substituted or unsubstituted C₂-C₁₄ alkynyl, substituted or unsubstituted C₁-C₁₄ acyl, or NR³R⁴;
R⁸, R⁹, R¹⁰, R¹¹, and R¹² are independently H, OH, halogen, substituted or unsubstituted C₁-C₂₀ alkyl, sub substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, substituted or unsubstituted C₁-C₂₀ acyl, or NR³R⁴, or wherein, as valence permits, R⁸ and R⁹, R⁹ and R¹⁰, R¹⁰ and R¹¹, or R¹¹ and R¹², together with the atoms to which they are attached, form a 3-10 membered substituted or unsubstituted cyclic moiety optionally including from 1 to 3 heteroatoms;
R² is H, OH, halogen, or substituted or unsubstituted C₁-C₄ alkyl; and
R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, or substituted or unsubstituted C₁-C₂₀ acyl.

7. The method of claim 1, wherein the carborane or carborane analog comprises a compound defined by Formula IX, or a pharmaceutically acceptable salt thereof 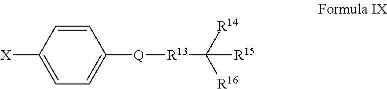
wherein
Q is a substituted or unsubstituted dicarba-closo-dodecaborane cluster, and 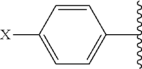
and R¹³ are attached to Q in a para configuration;
X is OH, NHR², SH, or S(O)(O)NHR²;
R¹³ is substituted or unsubstituted C₁-C₁₉ alkyl, substituted or unsubstituted C₂-C₁₉ alkenyl, substituted or unsubstituted C₂-C₁₉ alkynyl, or substituted or unsubstituted C₁-C₂₀ acyl; and
R¹⁴, R¹⁵, and R¹⁶ are independently hydrogen, halogen, hydroxyl, substituted or unsubstituted C₁-C₁₈ alkyl, substituted or unsubstituted C₂-C₁₈ alkenyl, substituted or unsubstituted C₁-C₁₈ alkynyl, substituted or unsubstituted C₂-C₁₈ aryl, substituted or unsubstituted C₃-C₁₈ cycloalkyl, substituted or unsubstituted C₁-C₂₀ acyl, or NR³R⁴, or wherein, as valence permits, R¹⁴ and R¹⁵, R¹⁴ and R¹⁶, or R¹⁵ and R¹⁶, together with the atoms to which they are attached, for a 3-10 membered substituted or unsubstituted cyclic moiety optionally including from 1 to 3 heteroatoms,
with the proviso that at least two of R¹⁴, R¹⁵ and R¹⁶ are not hydrogen, halogen, or hydroxyl; and
with the proviso that when X is OH and R¹³ is a C₅ alkyl, R¹⁴, R¹⁵, and R¹⁶ are not H, methyl, and methyl.

8. The method of claim 1, wherein the carborane or carborane analog comprises a compound defined by Formula XI, or a pharmaceutically acceptable salt thereof 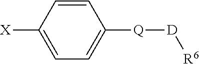
wherein
Q is a substituted or unsubstituted dicarba-closo-dodecaborane cluster;
D is —S—, —S(O)—, —S(OXO)—, —S(O)(NH)—, —P(O)(OH)O—, —P(O)(OH)NH—, or —O—;
X is OH, NHR², SH, or S(O)(O)NHR²;
R⁶ is substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₂-C₂₀ alkylheteroaryl, substituted or unsubstituted C₄-C₂₀ alkylcycloalkyl, or substituted or unsubstituted C₄-C₂₀ alkylheterocycloalkyl; and
R² is H, OH, halogen, or substituted or unsubstituted C₁-C₄ alkyl.

9. The method of claim 1, wherein the carborane or carborane analog comprises a compound defined by Formula XII, or a pharmaceutically acceptable salt thereof 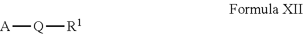
wherein
Q is a substituted or unsubstituted dicarba-closo-dodecaborane cluster, and A and R⁷ are attached to Q in a para configuration;
A is a substituted or unsubstituted heteroaryl ring;
R¹ is substituted or unsubstituted C₂-C₂₀ alkyl, substituted or unsubstituted C₂-C₂₀ alkenyl, substituted or unsubstituted C₂-C₂₀ alkynyl, substituted or unsubstituted C₃-C₂₀ alkylaryl, substituted or unsubstituted C₃-C₂₀ alkylheteroaryl, substituted or unsubstituted C₄-C₂₀ alkylcycloalkyl, substituted or unsubstituted C₄-C₂₀ alkylheterocycloalkyl, substituted or unsubstituted C₁-C₂₀ acyl, C₁-C₂₀ acyl, —C(O)NR³R⁴, —S(O)—R³, —S(O₂)—R³, substituted or unsubstituted C₂-C₂₀ heteroalkyl, or NR³R⁴; and
R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, and substituted or unsubstituted C₂-C₂₀ heteroalkyl.

10. The method of claim 9, wherein the carborane or carborane analog comprises a compound defined by Formula XIIA, or a pharmaceutically acceptable salt thereof 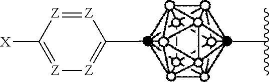
wherein
● is a carbon atom;
◯ is B—H, B-halogen, B-alkyl, B—OH, or B—NH₂;
X is OH, NHR², SH, or S(OXO)NHR²;
Z is, individually for each occurrence, N or CH, with the proviso that at least one of Z is N;
R¹ is substituted or unsubstituted C₂-C₂₀ alkyl, substituted or unsubstituted C₂-C₂₀ alkenyl, substituted or unsubstituted C₂-C₂₀ alkynyl, substituted or unsubstituted C₃-C₂₀ alkylaryl, substituted or unsubstituted C₃-C₂₀ alkylheteroaryl, substituted or unsubstituted C₄-C₂₀ alkylcycloalkyl, substituted or unsubstituted C₄-C₂₀ alkylheterocycloalkyl, substituted or unsubstituted C₁-C₂₀ acyl, C₁-C₂₀ acyl, —C(O)NR³R⁴, —S(O)—R³, —S(O₂)—R³, substituted or unsubstituted C₂-C₂₀ heteroalkyl, or NR³R⁴;
R² is H, OH, halogen, or substituted or unsubstituted C₁-C₄ alkyl; and
R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, and substituted or unsubstituted C₂-C₂₀ heteroalkyl.

11. The method of claim 10, wherein the carborane or carborane analog comprises a compound defined by one of the formulae below, or a pharmaceutically acceptable salt thereof: 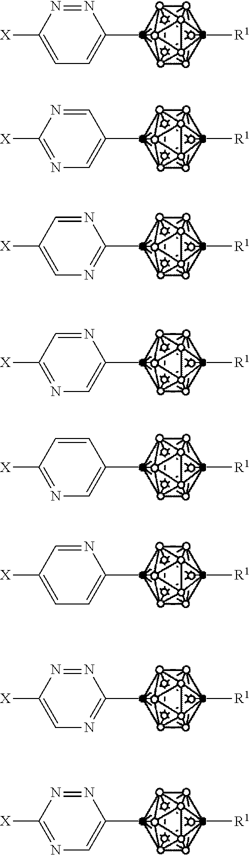
wherein
● is a carbon atom;
◯ is B—H, B-halogen, B-alkyl, B—OH, or B—NH₂;
X is OH, NHR², SH, or S(OXO)NHR²;
R¹ is substituted or unsubstituted C₂-C₂₀ alkyl, substituted or unsubstituted C₂-C₂₀ alkenyl, substituted or unsubstituted C₂-C₂₀ alkynyl, substituted or unsubstituted C₃-C₂₀ alkylaryl, substituted or unsubstituted C₃-C₂₀ alkylheteroaryl, substituted or unsubstituted C₄-C₂₀ alkylcycloalkyl, substituted or unsubstituted C₄-C₂₀ alkylheterocycloalkyl, substituted or unsubstituted C₁-C₂₀ acyl, C₁-C₂₀ acyl, —C(O)NR³R⁴, —S(O)—R³, —S(O₂)—R³, substituted or unsubstituted C₂-C₂₀ heteroalkyl, or NR³R⁴;
R² is H, OH, halogen, or substituted or unsubstituted C₁-C₄ alkyl; and
R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, and substituted or unsubstituted C₂-C₂₀ heteroalkyl.

12. The method of claim 10, wherein the carborane or carborane analog comprises a compound defined by one of Formula XIIB-XIIF, or a pharmaceutically acceptable salt thereof: 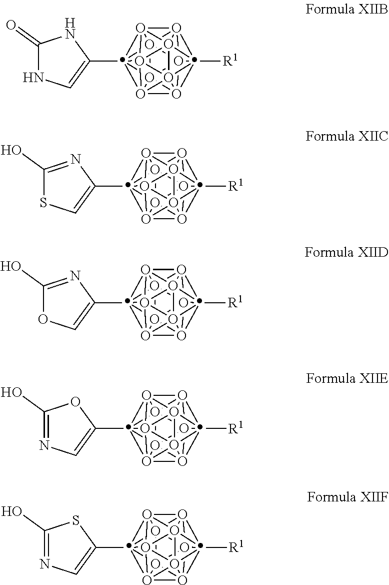
wherein
● is a carbon atom;
◯ is B—H, B-halogen, B-alkyl, B—OH, or B—NH₂;
R¹ is substituted or unsubstituted C₂-C₂₀ alkyl, substituted or unsubstituted C₂-C₂₀ alkenyl, substituted or unsubstituted C₂-C₂₀ alkynyl, substituted or unsubstituted C₃-C₂₀ alkylaryl, substituted or unsubstituted C₃-C₂₀ alkylheteroaryl, substituted or unsubstituted C₄-C₂₀ alkylcycloalkyl, substituted or unsubstituted C₄-C₂₀ alkylheterocycloalkyl, substituted or unsubstituted C₁-C₂₀ acyl, C₁-C₂₀ acyl, —C(O)NR³R⁴, —S(O)—R³, —S(O₂)—R³, substituted or unsubstituted C₂-C₂₀ heteroalkyl, or NR³R⁴;
R² is H, OH, halogen, or substituted or unsubstituted C₁-C₄ alkyl; and
R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, and substituted or unsubstituted C₂-C₂₀ heteroalkyl.

13. The method of claim 1, wherein the carborane or carborane analog comprises a compound defined by one of the formulae below, or a pharmaceutically acceptable salt thereof: 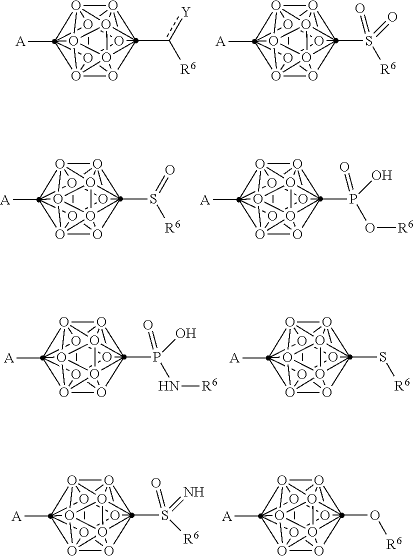
wherein
● is a carbon atom;
◯ is B—H, B-halogen, B-alkyl, B—OH, or B—NH₂;
the dotted line to Y indicates that the bond can be a single bond or a double bond, as valence permits;
A is a substituted or unsubstituted heteroaryl ring;
Y, when present, is O, halogen, OR^{2′}, NHR², SH, or S(OXO)NHR²;
R⁶ is substituted or unsubstituted C₁-C₁₉ alkyl, substituted or unsubstituted C₂-C₁₉ alkenyl, substituted or unsubstituted C₂-C₁₉ alkynyl, substituted or unsubstituted C₂-C₁₉ alkylaryl, substituted or unsubstituted C₂-C₁₉ alkylheteroaryl, substituted or unsubstituted C₄-C₁₉ alkylcycloalkyl, substituted or unsubstituted C₄-C₁₉ alkylheterocycloalkyl, and substituted or unsubstituted C₂-C₂₀ heteroalkyl, or NR³R⁴;
R² is H, OH, halogen, or substituted or unsubstituted C₁-C₄ alkyl;
R^{2′} is H or substituted or unsubstituted C₁-C₄ alkyl; and
R³ and R⁴ are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₂-C₂₀ alkylaryl, substituted or unsubstituted C₄-C₂₀ alkylcycloalkyl, and substituted or unsubstituted C₂-C₂₀ heteroalkyl.

14. (canceled)

15. (canceled)

16. The method of claim 1, wherein the carborane or carborane analog comprises a compound defined by Formula XIV, or a pharmaceutically acceptable salt thereof 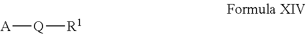
wherein
A is a substituted or unsubstituted aryl ring or a substituted or unsubstituted heteroaryl ring;
Q is a spacer group chosen from one of the following: 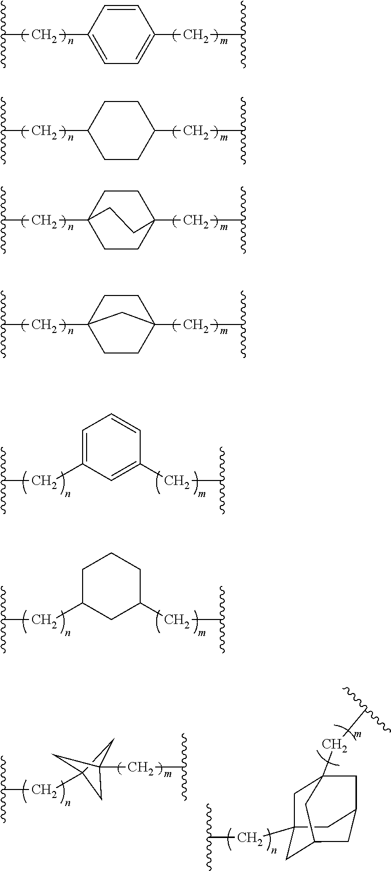
where m and n are each individually 0, 1, 2, or 3;
R¹ is substituted or unsubstituted C₄-C₂₀ alkyl, substituted or unsubstituted C₄-C₂₀ heteroalkyl, substituted or unsubstituted C₂-C₂₀ alkenyl, substituted or unsubstituted C₂-C₂₀ alkynyl, substituted or unsubstituted C₃-C₂₀ alkylaryl, substituted or unsubstituted C₃-C₂₀ alkylheteroaryl, substituted or unsubstituted C₄-C₂₀ alkylcycloalkyl, substituted or unsubstituted C₄-C₂₀ alkylheterocycloalkyl, substituted or unsubstituted C₁-C₂₀ acyl, C₁-C₂₀ acyl, —C(O)NR³R⁴, or NR³R⁴; and
R³ and R⁴ are independently selected from substituted or unsubstituted C₁-C₂₀ alkyl, substituted or unsubstituted C₁-C₂₀ heteroalkyl, substituted or unsubstituted C₂-C₂₀ alkenyl, substituted or unsubstituted C₂-C₂₀ alkynyl, substituted or unsubstituted C₂-C₂₀ alkylaryl, or substituted or unsubstituted C₄-C₂₀ alkylcycloalkyl.

17. (canceled)

18. (canceled)

19. (canceled)

20. (canceled)

21. (canceled)

22. The method of claim 1, wherein the carborane or carborane analog comprises WT-IV-012, the structure of which is shown below

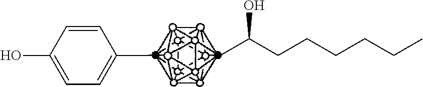

23. (canceled)

24. The method of claim 1, wherein the carborane or carborane analog has an EC₅₀ of 800 nM or less, less, at estrogen receptor beta (ERβ); wherein the carborane or carborane analog has an ERβ-to-ERα agonist ratio of 8 or more; or a combination thereof.

25. The method of claim 1, wherein the carborane or carborane analog comprises a selective ERs agonist.

26. (canceled)

27. The method of claim 1, wherein the lupus comprises systemic lupus erythematosus (SLE), cutaneous lupus erythematosus (CLE), drug-induced lupus, or neonatal lupus.

28. (canceled)

29. The method of claim 1, wherein the method further comprises administration of at least one additional therapeutic as part of the treatment regimen; wherein said additional therapeutic is selected from the group consisting of: corticosteroids, NSAIDS, antimalarials, immunomodulating agents, immunosuppressive agents, and anticoagulants.

30. (canceled)

31. The method of claim 29, wherein said additional therapeutic is a corticosteroid selected from the group consisting of prednisone, prednisolone, methylprednisolone, betamethasone, dexamethasone, triamcinolone and hydrocortisone.

32. (canceled)
